(19) United States
(12) Patent Application Publication
(10) Pub. No.: US 2024/0377403 A1
(43) Pub. Date: Nov. 14, 2024

(54) BIOMARKERS

(71) Applicant: THE UNIVERSITY OF BIRMINGHAM, Birmingham (GB)

(72) Inventors: Gary Middleton, Birmingham (GB); Akshay Patel, Birmingham (GB); Jonathan Michael Blackburn, Cape Town (ZA); Ti-Myen Tan, Kuala Lumpur (MY)

(21) Appl. No.: 18/692,762

(22) PCT Filed: Sep. 16, 2022
(86) PCT No.: PCT/GB2022/052359

§ 371 Date: Mar. 15, 2024

(30) Foreign Application Priority Data
Sep. 16, 2021 (GB) 2113264.2

(51) Int. Cl. G01N 33/574; G01N 33/564

(52) U.S. Cl. CPC G01N 33/57484; G01N 33/564

(57) ABSTRACT

A method of providing a prognosis to a subject, the method comprising: i. providing a biological sample obtained from the subject; ii. determining the level of a panel of biomarkers comprising or consisting of SPATA 19, SPACA3, TSPY3, TCEA2, TSGA10, and LUZP4 in the sample; iii. comparing the level of the panel of biomarkers in the sample with a reference level of the same panel of biomarkers; iv. using the results from (iii) to provide a prognosis to the subject. More particularly, a method of providing a prognosis to a subject with cancer.

### FIELD OF INVENTION

The invention relates to novel biomarkers and their use in providing a prognosis to a subject with non-small cell lung cancer (NSCLC). The invention also relates to pharmaceutical compositions for use in treating and/or preventing NSCLC.

### BACKGROUND

Worldwide, lung cancer is the leading cause of malignancy-related death in men and the second in women. Only 18% of patients at initial presentation are suitable for curative treatment, which is mainly surgical resection. The overall 5-year survival in treated patients is 20-30%, which can improve to 60% in early stage disease (1,2).

Despite this, a significant proportion of early-stage cancers relapse from aggressive disease within the first year post-operatively.

The behaviour of these cancers does not follow the disease patterns set out by prognostic scores such as the TNM staging system. Biologically, these early-stage cancers behave very differently and this therefore warrants the development of scoring systems that align more closely with the biological nature of these specific lung cancers, and which are therefore better able to prognose surgically resected disease.

Few prognostic biomarkers for early stage lung cancers have been described, and those that have are limited in their utility owing to the lack of proper validation and lack of adequate sensitivity and/or specificity (3).

Genomic biomarker investigation has advanced our understanding of lung cancer, and indeed mutations in p53, KRAS, eGFR and BRCA for example are well established aberrancies seen in early stage cancer and confer significantly reduced overall 5-year survival (4). However, assessing tissue level DNA requires access to tissue, and biopsies are not always truly representative of the tumour landscape owing to intra-tumoural heterogeneity (5,6). Multiplex gene panels in breast cancer such as MammaPrint and OncotypeDX similarly require fresh frozen tissue collected in RNA preservation solution which adds to the complexity (7).

Transcriptomic and epigenetic biomarker research is also in the early stages; JAK-STAT pathway mRNA is being explored as an NSCLC biomarker and associations are being made between global CpG methylation patterns and outcome in adenocarcinoma (9,10). However, these fields are still within the preliminary phases, and translation into the clinic is not without challenges, such as very high costs.

Furthermore, few proteomic signatures which stratify outcome in NSCLC have been validated. Mass spectrometry defined signatures have demonstrated poor survival in early stage lung cancer histological specimens, but despite this, the mechanistic relationship between proteomic signatures and disease outcome is poorly understood (4,11), and tissue samples are usually required.

There therefore remains a need for novel prognostic biomarkers for lung cancer, as well as new therapeutics.

### SUMMARY

In an aspect, the invention provides a method of providing a prognosis for a subject with cancer, the method comprising:
i. providing a biological sample obtained from the subject;
ii. determining the level of a panel of biomarkers comprising or consisting of SPATA19, SPACA3, TSPY3, TCEA2, TSGA10, and LUZP4 in the sample;
iii. comparing the level of the panel of biomarkers in the sample with a reference level of the same panel of biomarkers;
iv. using the results from (iii) to provide a prognosis to the subject.

The subject may be a subject that has been diagnosed with cancer. The cancer may be a lung cancer. The lung cancer may be NSCLC. The subject may be a human or non-human mammal.

The prognosis may be given pre or post treatment. The treatment may be a surgery, such as a resection.

The prognosis may be of likelihood of survival. The likelihood of survival may be given as the likelihood of surviving 5 years or more after the prognosis. The likelihood of survival may be given as the likelihood of surviving 5 years or more after the treatment.

The subject may be said to have a poor prognosis when the level of the panel of biomarkers is high. The poor prognosis may be a low likelihood of survival.

The subject may be said to have a good prognosis when the level of the panel of biomarkers is low. The good prognosis may be a high likelihood of survival.

The comparing step may be performed using an algorithm or piece of software for which a probability score can be generated. The probability may be of likelihood of survival.

The reference level of the same panel of biomarkers may be from an equivalent biological sample obtained from a patient who at 5 years post-surgical resection is determined to be cancer-free.

When the biomarker panel consists of SPATA19, SPACA3, TSPY3, TCEA2, TSGA10, and LUZP4, the level of the biomarkers is determined to be low when a ROC value of below around 40 to 45 is calculated, for example, below about 40, 41, 42, 43, 44, or 45. In an embodiment the ROC value is low if a value below 43.3 is calculated. The level of the panel is determined to be high when a ROC value of over around 40 to 45 is calculated, for example, over about 40, 41, 42, 43, 44, or 45. In an embodiment the ROC value is high if a value over 43.3 is calculated.

When the panel consists of SPATA19, SPACA3, TSPY3, TCEA2, TSGA10, and LUZP4, and a low likelihood of survival is prognosed, the subject may have a chance of survival of about 20%, in an embodiment about 19.9%.

The biomarker panel may further comprise or consist of one or more of, such as one of, two of, three of, four of, five of, six of, or all of GLS2, HMGN5, HDAC4, IMPDH1, TXN2, TFG, and PPP2R1A.

When the biomarker panel consists of SPATA19, SPACA3, TSPY3, TCEA2, TSGA10, LUZP4, GLS2, HMGN5, HDAC4, IMPDH1, TXN2, TFG, and PPP2R1A, the level of the biomarker panel is determined to be low when a ROC value of below around 60 to 70 is calculated, for example, below about 60, 61, 62, 63, 64, 65, 66, 67, 68, 69, or 70. In an embodiment the ROC value is low if a value below 66.96 is calculated. The level of the biomarker panel is determined to be high when a ROC value of over around 60 to 70 is calculated, for example, over about 60, 61, 62, 63, 64, 65, 66, 67, 68, 69, or 70. In an embodiment the ROC value is high if a value over 66.96 is calculated.

When the biomarker panel consists of SPATA19, SPACA3, TSPY3, TCEA2, TSGA10, LUZP4, GLS2, HMGN5, HDAC4, IMPDH1, TXN2, TFG, and PPP2R1A, and a low likelihood of survival is prognosed, the subject may have a chance of survival of around 5 to 10%, in an embodiment about 7.6%.

Alternatively, the biomarker panel may further comprise or consist of one or more of, such as one of, two of, three of, four of, five of, six of, seven of, eight of, nine of, or all of CTNNA2, MAGEB2, SPO11, MAGEB4, MAEL, CSAG1, MAGEB5, COX6B2, GAGE2, or TSSK6.

When the biomarker panel consists of SPATA19, SPACA3, TSPY3, TCEA2, TSGA10 LUZP4, CTNNA2, MAGEB2, SPO11, MAGEB4, MAEL, CSAG1, MAGEB5, COX6B2, GAGE2, and TSSK6, the level of the panel is determined to be low when a ROC value of below around 40 to 50 is calculated, for example, below about 40, 41, 42, 43, 44, 45, 46, 47, 48, 49, or 50. In an embodiment the ROC value is low if a value below 46.74 is calculated, and the level of the panel is determined to be high when a ROC value of above around 40 to 50 is calculated, for example, above about 40, 41, 42, 43, 44, 45, 46, 47, 48, 49, or 50. In an embodiment the ROC value is high if a value over 46.74 is calculated.

When the biomarker panel consists of SPATA19, SPACA3, TSPY3, TCEA2, TSGA10 LUZP4, CTNNA2, MAGEB2, SPO11, MAGEB4, MAEL, CSAG1, MAGEB5, COX6B2, GAGE2, and TSSK6, and a low likelihood of survival is prognosed, the subject may have a chance of survival of around 15 to 20%, in an embodiment about 16.4%.

Alternatively, the biomarker panel may further comprise or consist of one or more of, such as one of, two of, three of, four of, five of, six of, seven of, eight of, nine of, ten of, 11 of, 12 of, 13 of, 14 of, 15 of, 16 of, 17 of, 18 of, 19 of, 20 of, 21 of, 22 of, 23 of, 24 of, 25 of, 26 of, 27 of, 28 of, 29 of, 30 of, 31 of, 32 of, 33 of, 34 of, 35 of, 36 of, 37 of, 38 of, 39 of, 40 of, 41 of, 42 of, 43 of, 44 of, 45 of, 46 of, 47 of, 48 of, 49 of, 50 of, 51 of, 52 of, 53 of, or all of CASP7, GLS2, CTNNA2, ITPKB, AFF4, MAGEB2, C1orf174, TYRO3_int, DCBLD2, PCLAF, SPO11, BPIFA1, MAGEB4, HMGN5, MAEL, HDAC4, SOX15, HOOK1, CDK16, CSAG1, IMPDH1, MAGEB5, TXN2, NFYA, PHF7, HIST1H1C, IP6K1, TFG, AIM2, SGO1, PYCR1, FAM50B, HK2, ERBB3_int, TBL1X, ZNF207, EEF1D, PPP2R1A, MAP2K7, RPL7A, CBLC, COX6B2, ACTB, CA9, FLCN, GAGE2, ARAF, AK3, HMG20B, CNN1, EPAS1, EAPP, TSSK6, and GRK6.

The methods of the invention may be used, for example, for any one or more of the following: to diagnose a subject with lung cancer, such as NSCLC; to advise on the prognosis of a subject with lung cancer, such as NSCLC; to advise on treatment options for a subject with lung cancer, such as NSCLC, for example to treat current symptoms or to slow disease progression.

### Intervention

In another aspect, the invention provides a method of identifying a subject likely to benefit from treatment, the method comprising:
i. performing the method of the first aspect; and
ii. determining that a subject with a poor prognosis is likely to benefit from treatment.

The method may further comprise administering a therapeutic medication to the subject if the individual is given a poor prognosis. The treatment may be a more aggressive form of treatment, such as chemotherapy, radiotherapy, antibody therapy, T-cell therapy, or otherwise.

### Medication

A therapeutic or preventative medication referred to herein may comprise or consist of one or more of, such as one of, two of, three of, four of, five of, six of, seven of, eight of, nine of, ten of, 11 of, 12 of, 13 of, 14 of, 15 of, 16 of, 17 of, 18 of, 19 of, 20 of, 21 of, 22 of, 23 of, 24 of, 25 of, 26 of, 27 of, 28 of, 29 of, 30 of, 31 of, 32 of, 33 of, 34 of, 35 of, 36 of, 37 of, 38 of, 39 of, 40 of, 41 of, 42 of, 43 of, 44 of, 45 of, 46 of, 47 of, 48 of, 49 of, 50 of, 51 of, 52 of, 53 of, 54 of, 55 of, 56 of, 57 of, 58 of, 59 of, or 60 of SPATA19, CASP7, TSPY3, GLS2, TCEA2, CTNNA2, ITPKB, AFF4, MAGEB2, C1orf174, TSGA10, TYRO3_int, DCBLD2, PCLAF, SPO11, BPIFA1, MAGEB4, HMGN5, MAEL, LUZP4, HDAC4, SOX15, HOOK1, CDK16, CSAG1, SPACA3, IMPDH1, MAGEB5, TXN2, NFYA, PHF7, HIST1H1C, IP6K1, TFG, AIM2, SG01, PYCR1, FAM50B, HK2, ERBB3_int, TBL1X, ZNF207, EEF1D, PPP2R1A, MAP2K7, RPL7A, CBLC, COX6B2, ACTB, CA9, FLCN, GAGE2, ARAF, AK3, HMG20B, CNN1, EPAS1, EAPP, TSSK6, and GRK6.

The therapeutic or preventative medication may comprise or consist of one or more nucleic acid, such as DNA or mRNA, which encodes one or more of, such as one of, two of, three of, four of, five of, six of, seven of, eight of, nine of, ten of, 11 of, 12 of, 13 of, 14 of, 15 of, 16 of, 17 of, 18 of, 19 of, 20 of, 21 of, 22 of, 23 of, 24 of, 25 of, 26 of, 27 of, 28 of, 29 of, 30 of, 31 of, 32 of, 33 of, 34 of, 35 of, 36 of, 37 of, 38 of, 39 of, 40 of, 41 of, 42 of, 43 of, 44 of, 45 of, 46 of, 47 of, 48 of, 49 of, 50 of, 51 of, 52 of, 53 of, 54 of, 55 of, 56 of, 57 of, 58 of, 59 of, or 60 of SPATA19, CASP7, TSPY3, GLS2, TCEA2, CTNNA2, ITPKB, AFF4, MAGEB2, C1orf174, TSGA10, TYRO3_int, DCBLD2, PCLAF, SPO11, BPIFA1, MAGEB4, HMGN5, MAEL, LUZP4, HDAC4, SOX15, HOOK1, CDK16, CSAG1, SPACA3, IMPDH1, MAGEB5, TXN2, NFYA, PHF7, HIST1H1C, IP6K1, TFG, AIM2, SGO1, PYCR1, FAM50B, HK2, ERBB3_int, TBL1X, ZNF207, EEF1D, PPP2R1A, MAP2K7, RPL7A, CBLC, COX6B2, ACTB, CA9, FLCN, GAGE2, ARAF, AK3, HMG20B, CNN1, EPAS1, EAPP, TSSK6, and GRK6.

The therapeutic or preventative medication may comprise or consist of one or more nucleic acid, such as DNA or mRNA, which encodes an antibody which recognises one or more of, such as one of, two of, three of, four of, five of, six of, seven of, eight of, nine of, ten of, 11 of, 12 of, 13 of, 14 of, 15 of, 16 of, 17 of, 18 of, 19 of, 20 of, 21 of, 22 of, 23 of, 24 of, 25 of, 26 of, 27 of, 28 of, 29 of, 30 of, 31 of, 32 of, 33 of, 34 of, 35 of, 36 of, 37 of, 38 of, 39 of, 40 of, 41 of, 42 of, 43 of, 44 of, 45 of, 46 of, 47 of, 48 of, 49 of, 50 of, 51 of, 52 of, 53 of, 54 of, 55 of, 56 of, 57 of, 58 of, 59 of, or 60 of SPATA19, CASP7, TSPY3, GLS2, TCEA2, CTNNA2, ITPKB, AFF4, MAGEB2, C1orf174, TSGA10, TYRO3_int, DCBLD2, PCLAF, SPO11, BPIFA1, MAGEB4, HMGN5, MAEL, LUZP4, HDAC4, SOX15, HOOK1, CDK16, CSAG1, SPACA3, IMPDH1, MAGEB5, TXN2, NFYA, PHF7, HIST1H1C, IP6K1, TFG, AIM2, SGO1, PYCR1, FAM50B, HK2, ERBB3_int, TBL1X, ZNF207, EEF1D, PPP2R1A, MAP2K7, RPL7A, CBLC, COX6B2, ACTB, CA9, FLCN, GAGE2, ARAF, AK3, HMG20B, CNN1, EPAS1, EAPP, TSSK6, and GRK6.

The therapeutic or preventative medication may comprise or consist of one or more of, such as one of, two of, three of, four of, five of, or all of SPATA19, SPACA3, TSPY3, TCEA2, TSGA10, and LUZP4.

The therapeutic or preventative medication may comprise or consist of one or more nucleic acid, such as DNA or mRNA, which encodes one or more of, such as one of, two of, three of, four of, five of, or all of SPATA19, SPACA3, TSPY3, TCEA2, TSGA10, and LUZP4.

The therapeutic or preventative medication may comprise or consist of one or more nucleic acid such as DNA or mRNA which encodes an antibody which recognises one or more of, such as one of, two of, three of, four of, five of, or all of SPATA19, SPACA3, TSPY3, TCEA2, TSGA10, and LUZP4.

The therapeutic or preventative medication may comprise or consist of one or more of, such as one of, two of, three of, four of, five of, six of, seven of, eight of, nine of, ten of, 11 of, 12 of, or all of SPATA19, SPACA3, TSPY3, TCEA2, TSGA10, LUZP4, GLS2, HMGN5, HDAC4, IMPDH1, TXN2, TFG, and PPP2R1A.

The therapeutic or preventative medication may comprise or consist of one or more nucleic acid such as DNA or mRNA which encodes one or more of, such as one of, two of, three of, four of, five of, six of, seven of, eight of, nine of, ten of, 11 of, 12 of, or all of SPATA19, SPACA3, TSPY3, TCEA2, TSGA10, LUZP4, GLS2, HMGN5, HDAC4, IMPDH1, TXN2, TFG, and PPP2R1A.

The therapeutic or preventative medication may comprise or consist of one or more nucleic acid such as DNA or mRNA which encodes an antibody which recognises one or more of, such as one of, two of, three of, four of, five of, six of, seven of, eight of, nine of, ten of, 11 of, 12 of, or all of SPATA19, SPACA3, TSPY3, TCEA2, TSGA10, LUZP4, GLS2, HMGN5, HDAC4, IMPDH1, TXN2, TFG, and PPP2R1A.

The therapeutic or preventative medication may comprise or consist of one or more of, such as one of, two of, three of, four of, five of, six of, seven of, eight of, nine of, ten of, 11 of, 12 of, 13 of, 14 of, 15, or all of SPATA19, SPACA3, TSPY3, TCEA2, TSGA10, LUZP4, CTNNA2, MAGEB2, SPO11, MAGEB4, MAEL, CSAG1, MAGEB5, COX6B2, GAGE2, and TSSK6.

The therapeutic or preventative medication may comprise or consist of one or more nucleic acid, such as DNA or mRNA, which encodes one or more of, such as one of, two of, three of, four of, five of, six of, seven of, eight of, nine of, ten of, 11 of, 12 of, 13 of, 14 of, 15, or all of SPATA19, SPACA3, TSPY3, TCEA2, TSGA10, LUZP4, CTNNA2, MAGEB2, SPO11, MAGEB4, MAEL, CSAG1, MAGEB5, COX6B2, GAGE2, and TSSK6.

The therapeutic or preventative medication may comprise or consist of one or more nucleic acid such as DNA or mRNA which encodes an antibody which recognises one or more of, such as one of, two of, three of, four of, five of, six of, seven of, eight of, nine of, ten of, 11 of, 12 of, 13 of, 14 of, 15, or all of SPATA19, SPACA3, TSPY3, TCEA2, TSGA10, LUZP4, CTNNA2, MAGEB2, SPO11, MAGEB4, MAEL, CSAG1, MAGEB5, COX6B2, GAGE2, and TSSK6.

The therapeutic or preventative medication may be a formulated as a pharmaceutical composition.

In another aspect, the invention provides a pharmaceutical composition for use in treating and/or preventing lung cancer, such as NSCLC, comprising or consisting of a therapeutic or preventative medication referred to above.

Where the pharmaceutical composition comprises or consists of mRNA which encodes two or more antibodies, such antibodies may be encoded on a single piece of mRNA.

In another aspect, there is also provided a method of treating and/or preventing lung cancer, such as NSCLC, in a subject comprising: administering a therapeutically effective amount of a pharmaceutical composition of the invention to the subject.

The subject may be identified using a method of the invention. For example, the subject may have been given a poor prognosis using a method of the invention.

The skilled person will recognise that the medication or pharmaceutical composition can be administered, or arranged to be administered, at an appropriate dose via the appropriate administration route. The medication may comprise or consist of a therapeutically or prophylactically effective amount of the medication.

### The Sample

The biological sample may be a blood sample. The biological sample may be a serum or plasma sample.

The sample may be taken/obtained from the individual in the method of the invention. Alternatively, the sample may be provided (previously obtained, for example by a third party). The sample may be fresh, such as less than 1 day from withdrawal. Alternatively, the sample may be a stored sample, for example that has been frozen or refrigerated.

The sample, such as blood, may be taken pre-operatively such as before surgical resection, and then be used to prognose the subject's risk of recurrence/death after surgical resection. The sample, such as blood, may be taken post-operatively such as after surgical resection, and may then be used to prognose the subject's risk of recurrence/death after surgical resection.

Some or all of the steps of the method(s) of the invention may be carried out in vitro.

### Biomarker Determination

The presence, absence, or level of the panel of biomarkers of any method referred to herein may be determined by any suitable assay. The skilled person will recognise there are a number of methods and technologies available to determine the presence and/or level of the panel of biomarkers.

Determining the level of the panel of biomarkers may comprise quantifying the presence of the biomarkers in the panel in the sample. The level of panel of biomarkers in the sample may be compared relative to that of a control/reference sample, or a predetermined standard level. The reference sample may be a biological sample obtained from a patient who at 5 years post-surgical resection is alive and determined to be cancer-free. The reference sample may be a biological sample taken from a patient who is alive 5 or more years after being diagnosed with lunch cancer, such as NSCLC, and has had no recurrence of the cancer.

Determining the level of the panel of biomarkers may comprise binding each of the biomarkers in the panel with one or more probes. A method of the invention may further comprise detecting the binding of the probe(s) to each of the biomarkers in the panel, or detecting the level of bound probe-biomarker complexes.

Determining the level of the panel of biomarkers may comprise conducting an enzyme-linked immunosorbent assay (ELISA). The ELISA may comprise a competitive immunoassay, sandwich immunoassay or antibody capture. In particular an ELISA may be used to determine the level of one or more of the biomarkers in the panel in the sample. The ELISA may comprise a multiplexed ELISA. The multiplexed ELISA may comprise planar antibody arrays, Biochip Array Technology (BAT) multiplexed assay, membrane antibody arrays, or qualitative glass slide-based antibody arrays. The ELISA may be suspension-based, such as bead-based multiplex flow cytometry assay. The ELISA may be direct or indirect.

The level of the panel of biomarkers may be determined by aptamer-based ELASA (enzyme-linked apta-sorbent assay).

The level of the panel of biomarkers may be determined by western blot.

The level of the panel of biomarkers may be determined by detecting the marker directly, for example by mass-spectrometry. The mass-spectrometry may comprise liquid chromatography mass-spectrometry. The mass-spectrometry may comprise matrix assisted laser desorption ionization-time of flight mass-spectrometry (MALDI-TOF). The mass-spectrometry may comprise two-dimensional gel electrophoresis mass-spectrometry. The mass-spectrometry may comprise selective reaction monitoring mass-spectrometry. The mass-spectrometry may comprise tandem mass-spectrometry.

A probe referred to herein may be a binding agent that is capable of specific/selective binding to a protein biomarker of the panel of biomarkers. The binding agent may comprise or consist of a polypeptide and/or nucleic acid, such as DNA. The probe may comprise or consist of an antibody such as an autoantibody, an antibody variant or memetic, or a binding-fragment thereof. The probe may comprise or consist of an aptamer. The probe for a given biomarker of the panel may be a polyclonal or monoclonal antibody, or fragment thereof. The antibody, or fragment thereof, may be of any mammalian species, such as human, simian, porcine, camelid or rabbit.

The probe or probes may be immobilised on a substrate. One or more, or all of the probes may be anchored to a surface, such as the surface of a solid substrate. The solid substrate may be a plate, such as a microwell plate. The solid substrate may be a particle, such as a nano- or micro-particle. In one embodiment the solid substrate is a bead.

The probe may comprise a tag identification and/or capture. The tag may comprise a fluorescent molecule, or an enzyme. The probe may be radiolabelled.

In another aspect, there is provided a kit comprising probes capable of binding to each of the protein biomarkers of a panel of biomarkers described herein. Each probe may bind to an individual protein biomarker of the panel of biomarkers. The kit may contain a set of instructions.

Where ELISA, or similar assay, is used, the probe may be a primary antibody for binding to the target, and a secondary tagged-antibody probe may be provided for binding to the primary antibody or the biomarker for detection.

The level of the panel of biomarkers may refer to the protein level of each biomarker in the panel, which is detected in the sample.

The level of the panel of biomarkers may refer to the mRNA level encoding each biomarker in the panel, which is detected in the sample.

The level of the panel of biomarkers may refer to the level of autoantibodies which specifically recognise each biomarker in the panel, which are detected in the sample.

The autoantibodies may be detected using SEREX.

Autoantibody profiling is a promising approach that can incorporate the immune recognition of a myriad of aberrant cancer proteins into a single diagnostic test.

Autoantibodies (AAbs) reflect the initial humoral immune response against a tumour and their increased levels can be detectable months to years prior to clinical evidence of a primary tumour (12) or indeed recurrence post-resection of a primary tumour.

While the mechanisms involved in the production of AAbs in cancer patients (13) remain speculative, AAbs are well known to be sensitive biomarkers in the detection and surveillance of many types of tumours (13,14). Gnjatic and colleagues developed protein microarrays to assay the serological response of cancer patients to tumours (serological expression cloning, SEREX) [Gnjatic et al., 2009, J. Imm. Methods, 341:50-58]. These high-density protein microarrays, in which proteins are immobilised in their natural conformations, allow the functional testing of thousands of proteins simultaneously, increasing the chance of discovery of new autoantibody signatures (15).

Building on this work and principle, the inventors utilised the Sengenics Immunome™ Protein Array [Sengenics, Singapore] containing 1627 proteins, to screen sera from a total of 157 NSCLC patients. The inventors utilised a bespoke machine learning approach to investigate the utility of using pre-resection samples in the context of malignancy, to identify sera-based proteomic changes specifically associated with outcome in non-small cell lung cancer (NSCLC) following surgery. This yielded predictive biomarker panels which were able to reliably determine outcome in resected NSCLC patients with a high degree of accuracy. Such biomarkers, particularly cancer testis antigens (CTAGs), the expression of which is usually restricted yet have mechanistic links in various cancers, pose a viable therapeutic and prophylactic vaccine targets, especially in those subjects given a poor prognosis using a method of the invention.

Further, proteomic based research exploring autoantibodies in the serum poses an attractive option, as collecting serum in the pre-treatment phase is an easily implementable intervention and can be carried out in the clinic or bedside.

The panel of biomarkers of the invention is particularly useful in predicting survival in post-operative early stage lung cancer, which outperforms currently used autoantibody biomarkers in solid cancers.

Even further, CTAGs, which are biomarkers present in multiple panels referred to herein, trigger unprompted humoral immunity and immune responses in malignancies, altering tumour cell physiology and neoplastic behaviours. Their limited expression in normal somatic tissues coupled with recurrent up-regulation in epithelial carcinomas makes them highly attractive biomarker and vaccine targets.

### Definitions

A “prophylactically effective amount” interchangeable with ‘therapeutically effective amount’, or ‘effective amount’, or ‘therapeutically effective’, as used herein, refers to that amount which provides a therapeutic or preventative effect for a given condition and administration regimen. This is a predetermined quantity of active material calculated to produce a desired therapeutic effect in association with the required additive and diluent, i.e. a carrier or administration vehicle. Further, it is intended to mean an amount sufficient to reduce and most preferably prevent, a clinically significant deficit in the activity, function and response of the individual. Alternatively, a therapeutically effective amount is sufficient to cause an improvement in a clinically significant condition in an individual. As is appreciated by those skilled in the art, the amount of a compound may vary depending on its specific activity. Suitable dosage amounts may contain a predetermined quantity of active composition calculated to produce the desired therapeutic effect in association with the required diluent. In the methods and use for manufacture of compositions of the invention, a therapeutically effective amount of the active component is provided. A therapeutically effective amount can be determined by the ordinary skilled medical worker based on patient/individual characteristics, such as age, weight, sex, condition, complications, other diseases, etc., as is well known in the art.

The term “antibody” includes substantially intact antibody molecules, as well as chimeric antibodies, human antibodies, humanised antibodies (wherein at least one amino acid is mutated relative to the naturally occurring human antibodies), single chain antibodies, bispecific antibodies, antibody heavy chains, antibody light chains, homodimers and heterodimers of antibody heavy and/or light chains, and antigen binding fragments, antibody mimetics, and derivatives of the same. In particular, the term “antibody” as used herein refers to immunoglobulin molecules and immunologically active portions of immunoglobulin molecules, i.e., molecules that contain an antigen binding site that specifically binds an antigen, whether natural or partly or wholly synthetically produced. The term also covers any polypeptide or protein having a binding domain which is, or is homologous to, an antibody binding domain. These can be derived from natural sources, or they may be partly or wholly synthetically produced. Examples of antibodies are the immunoglobulin isotypes (e.g., IgG, IgE, IgM, IgD and IgA) and their isotypic subclasses; fragments which comprise an antigen binding domain such as Fab, scFv, Fv, dAb, Fd; and diabodies. Antibodies may be polyclonal or monoclonal. A monoclonal antibody may be referred to as a “mAb”.

It has been shown that fragments of a whole antibody can perform the function of binding antigens. Examples of binding fragments of the invention are (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment which consists of a VH domain; (v) isolated CDR regions; (vi) F(ab′)2 fragments, a bivalent fragment comprising two linked Fab fragments; (vii) single chain Fv molecules (scFv), wherein a VH domain and a VL domain are linked by a peptide linker which allows the two domains to associate to form an antigen binding site; (viii) bispecific single chain Fv dimers and; (ix) “diabodies”, multivalent or multispecific fragments constructed by gene fusion.

The biomarkers of the panel of biomarkers listed herein may include variants of the biomarker, for example variants having natural mutations/polymorphisms in a population. It is understood that reference to protein or nucleic acid “variants”, is understood to mean a protein or nucleic acid sequence that has at least 70%, 80%, 90%, 95%, 98%, 99%, 99.9% identity with the sequence of the fore mentioned protein or nucleic acid. The percentage identity may be calculated under standard NCBI blast p/n alignment parameters. “Variants” may also include truncations of a protein or nucleic acid sequence. Variants may include biomarker listed herein comprising the same sequence, but comprising or consisting of 1, 2, 3, 4, 5, 6, 7, 8, 9, or 10, or more modifications, such as substitutions, deletions, additions of nucleotides or bases. Variants may also comprise redundant/degenerate codon variations.

The skilled person will understand that optional features of one embodiment or aspect of the invention may be applicable, where appropriate, to other embodiments or aspects of the invention.

Embodiments of the invention will now be described in more detail, by way of example only, with reference to the accompanying drawings.

### BRIEF DESCRIPTION OF THE DRAWINGS

FIG. 1—shows a Multivariate Analysis using logistic and cox proportional hazards regression to assess outcome in terms of post-operative mortality and time to death respectively. Stepwise backward elimination was employed to remove the least significant independent predictors. All variables (age, gender, histology, IASLC stage, nodal status, lymphovascular invasion and adjuvant chemotherapy use) were entered into the model and successively removed depending on significance in the model.

FIG. 2—demonstrates a data analysis algorithm illustrating steps in raw data handling to applied machine learning processes and generation and testing of final biomarker panel in validation cohort. Throughout the algorithm, the number of biomarkers are indicated which are successively eliminated based on variable importance and stability in the model. At each iterative step, the number of biomarkers is displayed indicating successive removal and refinement of the model.

FIG. 3—shows ROC metrics displayed for additive modelling of biomarkers in the RFE set (n=60). There is a progressive linear increase in AUC (solid line starting in the middle), sensitivity (solid line starting at the top) and specificity (solid line starting at the bottom) with each cumulative addition of biomarkers. The solid lines show the interval change in each metric with each biomarker addition and the dashed lines represent the overall trend. There is a progressive improvement in all parameters up to 44 biomarkers, beyond which, there is a decay in performance for each metric as shown by the sharp negative deflection (arrow). The performance of the model deteriorated beyond 44 cumulative biomarkers indicating no added value.

FIG. 4—shows ROC curves demonstrating performance of Panel A (13 biomarkers) in both cohort 1 (test) and cohort 2 (validation). P value indicates no significant difference in performance of this model between cohorts. AUC 95% confidence intervals displayed in brackets.

FIG. 5—shows ROC curves demonstrating performance of Panel A (13 biomarkers) and Panel B (16 CTAG biomarkers from RFE set) in cohort 1 (test) (left panel) and cohort 2 (validation) (right panel). AUC confidence intervals displayed in brackets. P values indicate significant differences in performance in cohort 1 but not cohort FIG. 6—shows ROC curves demonstrating performance of Panel A (13 biomarkers) and Panel C (6 CTAG biomarkers from Panel A) in cohort 1 (test) (left panel) and cohort 2 (validation) (right panel). AUC confidence intervals displayed in brackets. P values indicate significant differences in performance in cohort 1 but not cohort 2.

FIG. 7—shows Kaplan Meier Survival Analysis for Panel A (13 biomarkers), Panel B (16 CTAG biomarkers from RFE set) and Panel C (6 CTAG biomarkers from panel A). All curves demonstrate significantly worse outcome in patients with high global expression of each biomarker signature. The number of patients in each expression group is shown in brackets in the legend for each Kaplan-Meier plot. Percentage survival out to 5 years is displayed to the right hand side for each expression group in each plot. The median survival for high expressers of Panel A, B and C is 479 days, 558 days and 600.5 days respectively.

FIG. 8—shows Multivariate Cox Proportional Hazards modelling in entire NSCLC cohort (n=157). Stepwise backward elimination was employed to remove the least significant independent predictors.

### MATERIALS AND METHODS

### Study Design

A total of 157 study participants' (NSCLC Stage I-III) pre-operative serum samples were utilised in the proteomics analysis. This was determined using a power calculation based on the standard deviations of each protein in the immunome array and sample size estimates were calculated across a range of power values (90-99%), finally settling on a power value of 95%. Once this overall cohort size was determined, a random set of patients was selected in order to train the machine learning model and subsequently tune the model hyperparameters using k-fold cross validation (16). This training cohort is known as cohort 1. A smaller independent, separate cohort of patients was selected to provide an unbiased evaluation of the final model. This separate cohort used to validate the model is known as cohort 2. Cohort sizes were determined using a stratified random sample-based approach to split the overall dataset. For reasonable sized datasets (n>100), this commonly used approach in machine learning settings has been shown to be close to optimal when allocating 66-70% of the samples to the training set (cohort 1) (17).

### Study Cohorts

Cohort 1 consisted of 111 NSCLC patients (65 survivors, 46 non-survivors). Cohort 2 consisted of 46 NSCLC patients (27 survivors, 19 non-survivors). Survivors were defined as patients who were alive and recurrence-free at follow-up. Median follow-up of the entire recurrence free population was 1825 days (range of 1195-2555 days). Non-survivors were defined as patients who died from post-operative recurrence within 12 months. The participant characteristics are summarised in Table 1. There was no significant difference between cohorts 1 and 2 in terms of age, gender and stage. There was a higher preponderance of adenocarcinomas in cohort 2 (60.9% versus 49.5%), and a higher preponderance of squamous cell carcinomas in cohort 1 (50.5 versus 39.1%).

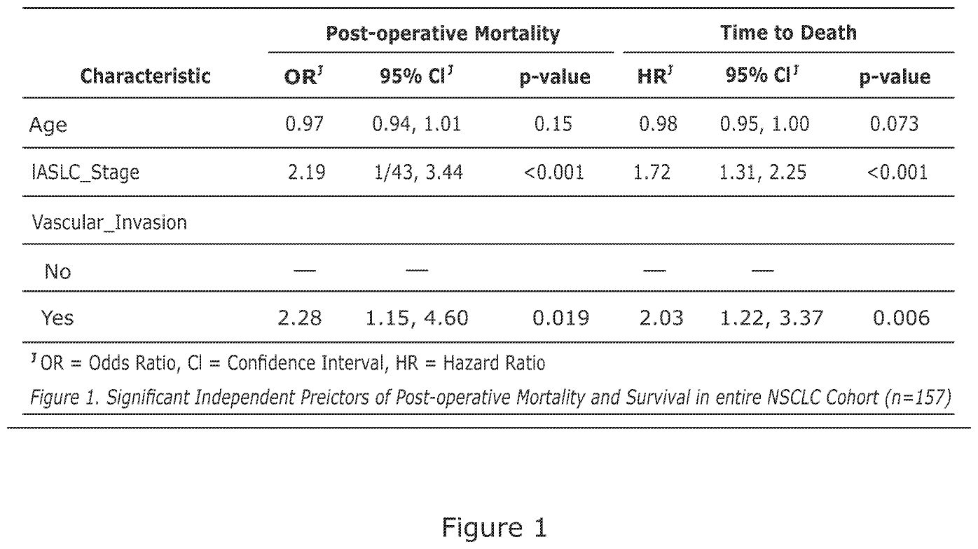

Survival distribution of the total study population is displayed in FIG. 1. Cox Proportional Multivariate Hazards Analysis identified IASLC stage and the presence of Lymphovascular invasion as significant independent negative prognostic risk factors (HR 1.72, p<0.001 and HR 2.03, p=0.006 respectively), however histology was not deemed significant. All samples were assayed using the Sengenics Immunome Protein Array containing 1600+ proteins spotted in quadruplicates (Sengenics, Singapore).

### Study Participants

Collaborating clinicians and principal researchers recruited the study group across two major tertiary sub-specialty centres in the midlands regions of England, UK. Patients underwent curative NSCLC resection at two major thoracic surgical units in England. Patients who had any other previous malignancy were excluded. All participants provided informed consent to participate in this study, previously approved by the West Midlands—Solihull Research Ethics Committee (Cancer of the Lung Biomarkers (CLUB): REC reference: 04/Q2704/34). The study had National Cancer Research Network (NCRN) approval and was an NCRN portfolio study. Patients were diagnosed by routine pathological examination of their excised primary tumour and staged according to the TNM staging system for NSCLC according to the International Association for the Study of Lung Cancer (IASLC) guidelines (8th Edition) (54).

### Sample Collection

Serum samples were taken at enrolment or prior to surgery. Samples were collected from all participants in a starved state to maintain uniformity. A sample of 7 ml whole venous blood was taken into standard collection tubes and allowed to clot for 2 h. Samples were centrifuged at 3000G for 20 minutes. Serum was then carefully aspirated, divided into aliquots and stored at −80° C. (55).

### Protein Immunome Auto-Antibody Assay

Serum samples were thawed, mixed by vortexing and any precipitate was pelleted by centrifugation (13,000G, for 3 minutes). Aliquot of each sample (11.25 L) were then diluted 400-fold into Serum Assay Buffer (SAB; 0.1% v/v Triton, 0.1% w/v BSA in PBS; 20° C.), giving a final volume of 4.5 ml. 38 Replica Immunome protein array slides were removed from storage buffer and washed in 200 ml cold SAB on an orbital shaker (50RPM, 5 minutes). Each slide was then placed array side up in a hybridization chamber and incubated with individual diluted sera (4.5 mL) on a horizontal shaker for 2 hours at 20° C., with gentle agitation. Each protein array slide was then rinsed briefly twice with 30 mL SAB, followed by immersion in 200 mL of SAB buffer for 20 minutes at room temperature with gentle agitation. Each slide was then incubated with detection antibody (20 μg/ml Cy3-labelled anti-human IgG in SAB) for 2 hours at room temperature with gentle agitation, rinsed briefly with SAB buffer then washed three times in SAB for 5 minutes at room temperature. Excess buffer was removed by immersing the slide briefly in 200 mL deionised water, after which slides were then dried by centrifugation (240G for 2 minutes) at room temperature. Slides were then stored at room temperature and scanned the same day at 10 μm resolution using an Agilent G2505C fluorescence microarray laser scanner.

### Bioinformatics Analysis

Data pre-processing—Scanned images were pre-processed and quality control checks were performed on the generated data using the Sengenics internal pipeline (56). Composite normalization of the data was subsequently performed by using both quantile-based and intensity-based modules on the Cy3-labelled biotinylated BSA positive control probes as reported by Duarte et al (57). Autoantibody binding towards specific proteins were presented as relative fluorescent intensities (RFU) and used as inputs for downstream analysis.

Penetrance fold change analysis—The penetrance fold change (pFC) analysis compares both the frequency and strength of autoantibody signals with the intention of identifying biomarkers which are highly elevated in survivors. To achieve this, individual fold changes of survivors and non-survivors were estimated using the equation below:

$$IFC ( protein A , sample X ) = RFU ( protein A , sample X ) / μ RFU ( protein A , control group )$$

Protein A represents each protein in the Immunome array and X represent every sample assayed in the microarray platform. The mean RFU value for each protein in the control group were used as a background threshold.

For both the survivor and non-survivor groups respectively, pFC values for each group were obtained by calculating the mean IFC of patients which passes the IFC threshold of ≥2. The penetrance frequencies were then calculated by estimating the number of patients (in each group) which has an IFC≥2 [3]. Biomarkers were further filtered based on the criteria of i) pFC of survivors≥2, ii) % penetrance frequency of survivors≥10% and iii) penetrance frequency of non-survivors≤10%.

Selection of Biomarker Panel—A combination of feature selection and machine learning methodologies were used to determine the optimal number of biomarkers that were able to provide the best stratification between survivors and non survivors (58). For feature selection, univariate statistical tests, random forest importance and mutual information metrics were the filter methods used to rank biomarkers (The full list of filter functions are listed in Table 3). Given the degree of multi-collinearity between the biomarkers, Recursive-Feature Elimination (RFE) with Random Forest modelling was applied to the dataset, looping across 100 unsupervised iterations using random seeds for marker reliability. The top most stable biomarkers were used to generate biomarker panels by additively selecting the top-ranking biomarkers (top 3.75% of biomarkers, n=60) in a cumulative fashion, starting with the most stable biomarker from the RFE set (i.e. 1st, 1st+2nd, 1st+2nd+3rd etc). ROC metrics were determined for each additive model and the top-performing combination taken forward as input to machine learning models. Any further addition of biomarkers did not lead to significant improvements of model performance but only further increases in computational time. To determine the biomarker panel performance, ROC, sensitivity and specificity were evaluated and the biomarker panel with the best sensitivity and specificity was deemed the optimal panel to stratify between survivors and non-survivors. For this analysis, Boosted Logistic Regression was performed under default settings using accuracy estimation methods, repeated cross-fold validation and leave-one out cross validation (LOOCV) (59).

Model Selection—To corroborate marker selection from the RFE algorithm, lasso regression with repeated tenfold cross-validation in the training set was used. This was applied using the R package glmnet. The elastic-net penalty, α, that bridges the gap between lasso (α=1, the default) and ridge regression (α=0), to 0.9 for numerical stability (60) was set. Furthermore, proteomics data was processed using DESeq2 (v.4.0.2) software to identify differentially expressed proteins between survivors and non-survivors. A cut-off of gene-expression fold change of ≥2 or ≤0.5 and an FDR q≤0.05 was applied to select the most differentially expressed proteins.

Akaike Information Criterion—A model averaging approach using the Akaike information criterion (AIC) weights (57,61) was adopted in order to estimate the in-sample prediction error and thereby the relative quality of the statistical models for a given set of data. An information theoretics approach was used to calculate the AIC for each model permutation within the top ranking biomarkers using the glmulti and MuMIn packages in order to determine the most parsimonious model with the greatest explanatory predictive power. The AIC is a measure of how well a model fits the data relative to the other possible models given the data analysed and favours fewer parameters (62). The model with the lowest AIC is the best model approximating the outcome of interest. AIC can be expressed as:

$$AIC = - 2 ( loglikelihood ) + 2 K ,$$

K=number of model parameters and log-likelihood is a measure of model fit. In this study, as n/K≤60 for sample size n and the model with the largest value of K, the second-order bias correction version of the AIC (AICc) was used:

$$\begin{matrix}{AIC}_{c} = - 2 ( loglikelihood ) + 2 K + 2 K ( K + 1 ) n - K - 1 , \\ {AIC}_{c} = AIC + 2 K ( K + 1 ) n - K - 1 ,\end{matrix}$$

where n=sample size, K=number of model parameters and log-likelihood is a measure of model fit (61,63). From an information-theoretic perspective, the Akaike weights for a particular model can be regarded as the probability or “weight of evidence” that the model is the best model (in a Kullback-Leibler sense of minimizing the loss of information when approximating full reality by a fitted model) out of all of the models considered/fitted based on the available data set (61,62).

Pathway analysis—Biological process pathway analysis was carried out using Gene Ontology and PANTHER25. UniProt accession numbers of proteins corresponding to the biomarkers selected from RFE were uploaded to http://geneontology.org and all Homo sapiens genes in the database were used as a reference list. Fisher's exact with false discovery rate (FDR) multiple test correction was used for determining pathway significance.

### Additional Statistical Analyses

All other statistical analyses were done using the RFU values of 1600+ proteins using the R platform. ROC analyses were performed using the package OptimalCutpoints (64) and plotted using ggplot2 (65). Survival analyses were performed using survminer package (66). Machine learning analyses were performed using the mlr (67), party (68), ranger (59), randomForest (69) and praznik (70) and caret package. Power calculations were performed using the samplesize and sizepower packages (18,71,72). Data presentation in table format was implemented using the gtsummary package.

TABLE 1
cohort characteristics
Table 1. Clinicopathological Characteristics of the Study Cohorts

| | | |
|---|---|---|
| Group | Cohort 1 | Cohort 2 |

| Total Cohort Number | 111 | | 46 | |
|---|---|---|---|---|
| Male, n (%) | 63 | (56.8%) | 28 | (60.9%) |
| Female, n (%) | 48 | (43.2%) | 18 | (39.1%) |

| | | |
|---|---|---|
| Mean Age +/− SD (years) | 72.4 +/− 10.4 | 73.5 +/− 7.87 |

| | | | | |
|---|---|---|---|---|
| Adenocarcinoma, n (%) | 55 | (49.5%) | 28 | (60.9%) |
| Squamous Cell Carcinoma, n (%) | 56 | (50.5%) | 18 | (39.1%) |

Tumour Size, n (%)

| | | | | |
|---|---|---|---|---|
| T1 (0-3 cm) | 56 | (50.5%) | 16 | (34.8%) |
| T2 (3-5 cm) | 34 | (30.6%) | 21 | (45.7%) |
| T3 (5-7 cm) | 15 | (13.5%) | 4 | (8.7%) |
| T4 (>7 cm) | 6 | (5.4%) | 5 | (10.8%) |

Nodal Status, n (%)

| N0 | 74 | (66.7%) | 34 73.9%) | |
|---|---|---|---|---|
| N1 | 21 | (18.9%) | 7 | (15.2%) |
| N2 | 16 | (14.4%) | 5 | (10.9%) |
| Presence of Lymphovascular Invasion, n (%) | 52 | (46.8%) | 20 | (43.5%) |

IASLC Stage, n (%)

| | | | | |
|---|---|---|---|---|
| I | 71 | (64.0%) | 30 | (65.2%) |
| II | 17 | (15.3%) | 10 | (21.7%) |
| III | 22 | (19.8%) | 5 | (10.9%) |
| IV | 1 | (0.9%) | 1 | (2.2%) |
| Adjuvant Therapy, n (%) | 40 | (36.0%) | 18 | (39.1%) |
| Mortality, n (%) | 46 | (41.4%) | 19 | (41.3%) |
| Recurrence, n (%) | 46 | (41.4%) | 19 | (41.3%) |

| | | |
|---|---|---|
| Median Follow-up (days) | 1825 | 1805 |

TABLE 2
Biomarkers in Panel A

| | | Reference linking |
|---|---|---|
| | | to function and |
| Biomarker | Name | cancer |
| SPATA19 [symbol] | Spermatogenesis-associated protein 19 | (1-4) |
| TSPY3 [symbol] | Cancer Testis Antigen 78/Testis Specific Protein Y- | (5-11) |
| | Linked 3 | |
| GLS2 | Glutaminase 2 | |
| TCEA2 [symbol] | Transcription Elongation Factor A2/TFIIS/Testis- | (12-14) |
| | Specific SII gene | |
| TSGA10 [symbol] | Testis-specific gene protein 10/Cancer Testis | (15-18) |
| | Antigen 79 | |
| HMGN5 | High Mobility Group Nucleosome Binding Domain | (19-27) |
| | 5/NSBP1 | |
| LUZP4 [symbol] | Leucine Zipper Protein 4/Cancer Testis Antigen 28 | (28-30) |
| HDAC4 | Histone Deacetylase 4 | (31-34) |
| SPACA3 [symbol] | Sperm Acrosome membrane-associated protein | (35-37) |
| | 3/Cancer Testis Antigen 54 | |
| IMPDH1 | Inosine Monophosphate Dehydrogenase 1/LCA11 | (38-40) |
| TXN2 | Thioredoxin 2/MT-TRX/COXPD29 | (41) |
| TFG | Trafficking from ER to Golgi Regulator/TRKT3 | (42, 43) |
| | Oncogene/TRK-Fused Gene Protein | |
| PPP2R1A | Protein Phosphatase 2 Scaffold Subunit | (44-50) |
| | alpha/Serine Threonine Protein Phosphatase 2A | |

TABLE 3
List of filter functions applied to
determine the optimal biomarker panel

| Name | Test | R package |
|---|---|---|
| anova.test | univariate statistical test | mlr |
| auc | univariate statistical test | mlr |
| kruskal.test | univariate statistical test | mlr |
| party_cforest.importance | random forest importance | party |
| praznik_CMIM | mutual information | praznik |
| praznik_JMI | mutual information | praznik |
| praznik_JMIM | mutual information | praznik |
| praznik_MIM | mutual information | praznik |
| praznik_MRMR | mutual information | praznik |
| praznik_NJMIM | mutual information | praznik |
| praznik_DISR | mutual information | praznik |
| randomForest_importance | random forest importance | randomForest |
| ranger_permutation | random forest importance | ranger |
| ranger_impurity | random forest importance | ranger |
| variance | feature variance | mlr |

TABLE 4
Top most stable biomarkers as determined
by RFE (In order of importance)
S2. Top most stable biomarkers as determined
by RFE (In order of importance)

| | Repeated k-fold | | Lasso | |
|---|---|---|---|---|
| Biomarker | cross validation | LOOCV | Regression | DESeq 2 |
| SPATA19 | [symbol] | [symbol] | | |
| CASP7 | [symbol] | [symbol] | | |
| TSPY3 | [symbol] | [symbol] | | |
| GLS2 | [symbol] | [symbol] | [symbol] | [symbol] |
| TCEA2 | [symbol] | [symbol] | | |
| CTNNA2 | [symbol] | [symbol] | | [symbol] |
| ITPKB | [symbol] | [symbol] | | |
| AFF4 | [symbol] | [symbol] | | |
| MAGEB2 | [symbol] | [symbol] | [symbol] | |
| C1orf174 | [symbol] | [symbol] | [symbol] | |
| TSGA10 | [symbol] | [symbol] | | |
| TYRO3_int | [symbol] | | [symbol] | |
| DCBLD2 | [symbol] | | [symbol] | [symbol] |
| PCLAF | [symbol] | | | |
| SPO11 | [symbol] | [symbol] | | |
| BPIFA1 | [symbol] | [symbol] | | |
| MAGEB4 | [symbol] | [symbol] | [symbol] | |
| HMGN5 | [symbol] | | [symbol] | |
| MAEL | [symbol] | | | |
| LUZP4 | [symbol] | | [symbol] | |
| HDAC4 | [symbol] | | | |
| SOX15 | [symbol] | | | |
| HOOK1 | [symbol] | | | |
| CDK16 | [symbol] | | [symbol] | |
| CSAG1 | [symbol] | | | |
| SPACA3 | [symbol] | | | |
| IMPDH1 | [symbol] | | | |
| MAGEB5 | [symbol] | | | |
| TXN2 | [symbol] | | [symbol] | [symbol] |
| NFYA | [symbol] | | | |
| PHF7 | [symbol] | | [symbol] | [symbol] |
| HIST1H1C | [symbol] | | | |
| IP6K1 | [symbol] | | | |
| TFG | [symbol] | | | |
| AIM2 | [symbol] | | | |
| SGO1 | [symbol] | | | |
| PYCR1 | [symbol] | | | |
| FAM50B | [symbol] | | | |
| HK2 | [symbol] | | | |
| ERBB3_int | [symbol] | | | |
| TBL1X | [symbol] | | [symbol] | |
| ZNF207 | [symbol] | | [symbol] | |
| EEF1D | [symbol] | | | |
| PPP2R1A | [symbol] | | | |
| MAP2K7 | [symbol] | | | |
| RPL7A | [symbol] | | | [symbol] |
| CBLC | [symbol] | | | |
| COX6B2 | [symbol] | | | |
| ACTB | [symbol] | | | |
| CA9 | [symbol] | | | |
| FLCN | [symbol] | | | |
| GAGE2 | [symbol] | | | |
| ARAF | [symbol] | | | |
| AK3 | [symbol] | | | |
| HMG20B | [symbol] | | | |
| CNN1 | [symbol] | | | |
| EPAS1 | [symbol] | | | |
| EAPP | [symbol] | | [symbol] | [symbol] |
| TSSK6 | [symbol] | | | |
| GRK6 | [symbol] | | | |

### Examples

### Identification of Predictive Biomarkers

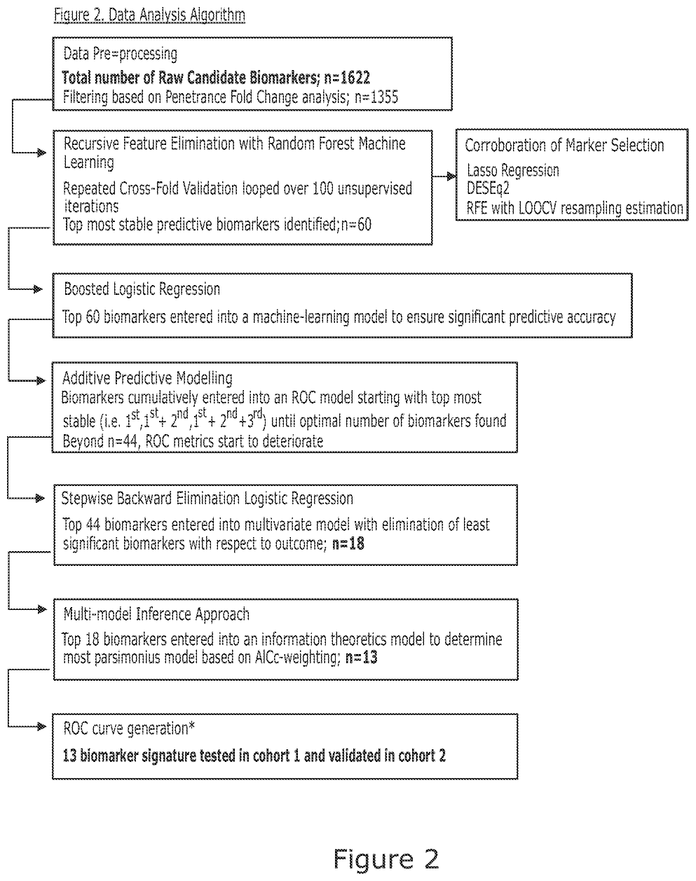

The final biomarker panel was selected on the basis of an iterative applied machine learning pipeline as specified in the methodology, the algorithm for which is shown in FIG. 2. This bespoke algorithm comprised machine learning elements that have been validated in other cancer classification datasets (18,19).

Initial data processing involved filtering according to the penetrance fold change analysis in order to avoid biasing subsequent model generation. 1355 biomarkers remained which were taken forward into the deeper analysis. Within the remaining biomarker data, >93% displayed collinearity of r>0.75 on Spearman Rank Correlation analysis, hence the reason for proceeding with recursive feature elimination by random forest modelling.

The biomarkers, which appeared most frequently with the highest importance values across 100 randomly seeded iterations, were subject to corroborative regression and genomics analysis methods, which indicate the biomarkers which were common to all analytical techniques. Overall, 60 biomarkers (referred therein as ‘RFE’ set) were identified as the most stable with no improvement in predictive performance beyond this number. The final panel (panel A) of biomarkers comprised 13 protein antigens which are listed in Table 2 (SPATA19, TSPY3, GLS2, TCEA2, TSGA10, HMGN5, LUZP4, HDAC4, SPACA3, IMPDH1, TXN2, TFG and PPP2R1A), also referred to herein as ‘Panel A’. A preponderance of bona-fide Cancer Testis Antigens (CTAGs) was noted in the RFE biomarker set (16/60 (26.7%)). Two further CTAG specific panels were explored in order to determine the prognostic relevance of these highly conserved proteins in NSCLC. ‘Panel B’ refers to the CTAGs extracted from the RFE set (16 biomarkers) and ‘Panel C’ refers to the CTAGs extracted from Panel A (6 biomarkers).

### Additive Predictive Modelling

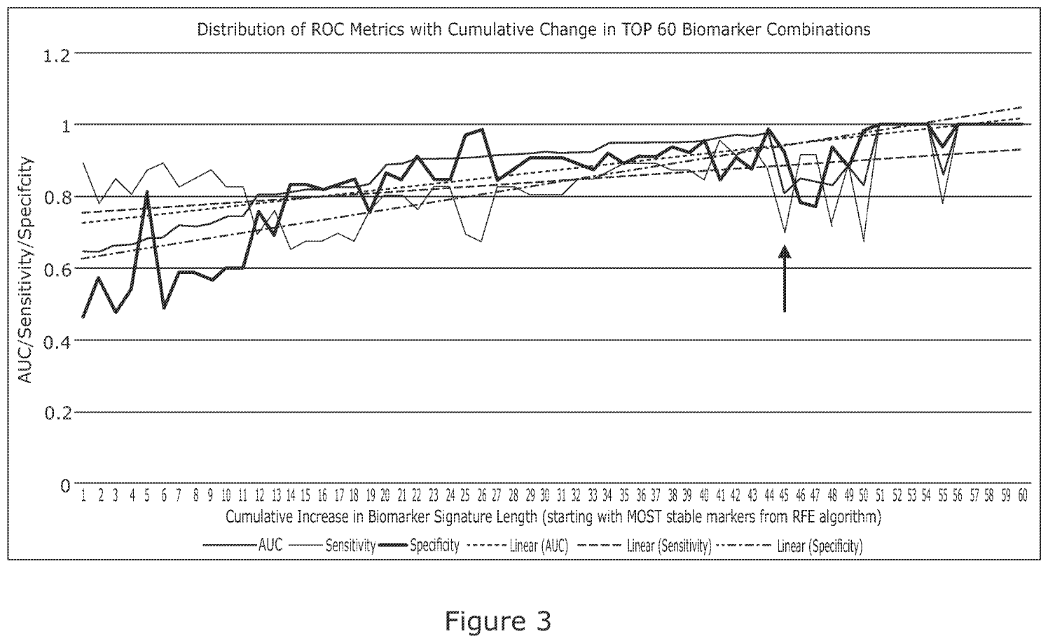

The RFE set of biomarkers was used to generate biomarker panels by additively selecting the top-ranking biomarkers in a cumulative fashion. These inputs were used to determine the ROC metrics at each additive iteration for cohort 1 (FIG. 3). An upward linear trend in all three parameters (AUC<sensitivity, specificity) was noted as more biomarkers were added. This progressive increase peaked at 44 cumulative biomarkers (AUC 0.44-0.975; Sensitivity 67.4%-87%; Specificity 46.2%-98.5%). Beyond this, the predictive metrics become more unstable and less uniform hence the decision to proceed with the top 44 biomarkers for deeper analysis.

### Multi-Model Inference Approach

Given that a 60-biomarker diagnostic scoring system would be cumbersome and impractical, an information-theoretic approach was used to determine the biomarker combination with the highest diagnostic potential in the most parsimonious model. Akiake Information Criterion method (AICc) was employed in order to estimate the “goodness of fit” of statistical models and thereby compare multiple models with one another. The AICc avoids overfitting the model in smaller sample sizes. Based on the cumulative ROC analysis, the top 44 biomarkers were proceeded with in this downstream analysis. Following stepwise backward elimination of these markers in a multivariate logistic regression model, with survivorship as the dependent variable, 18 biomarkers were determined to be the most significant and were therefore used in the multi-model inference analysis. Any further addition of more biomarkers did not lead to significant improvements of model performance but did contribute to significant increases in computational time.

### Assessing Model Performance

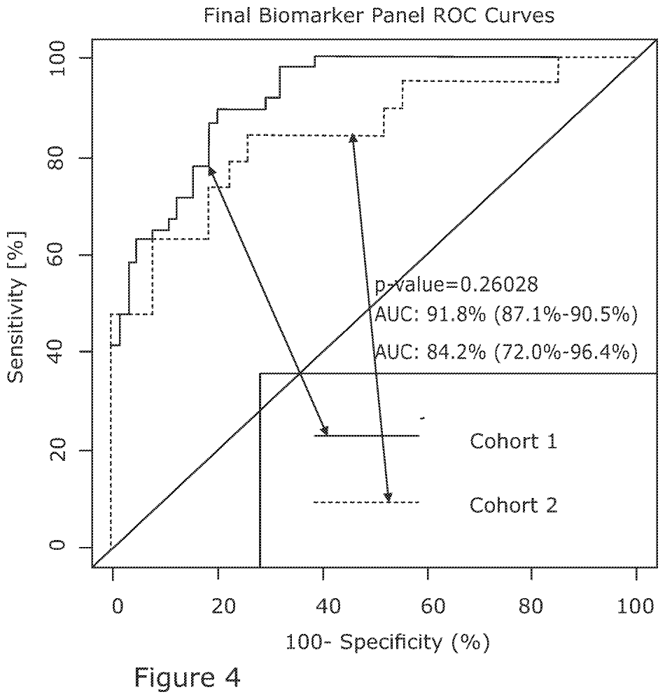

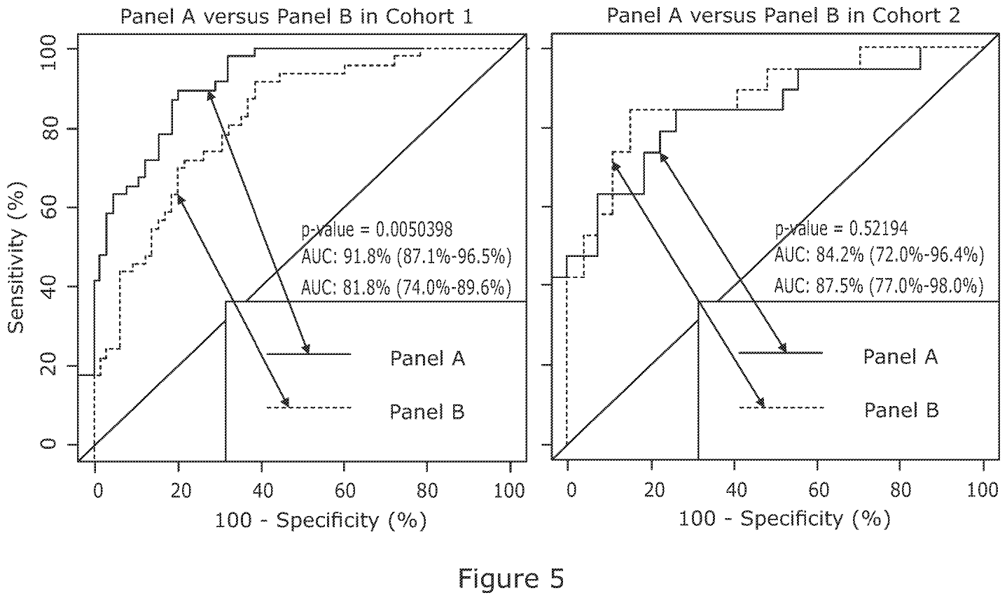

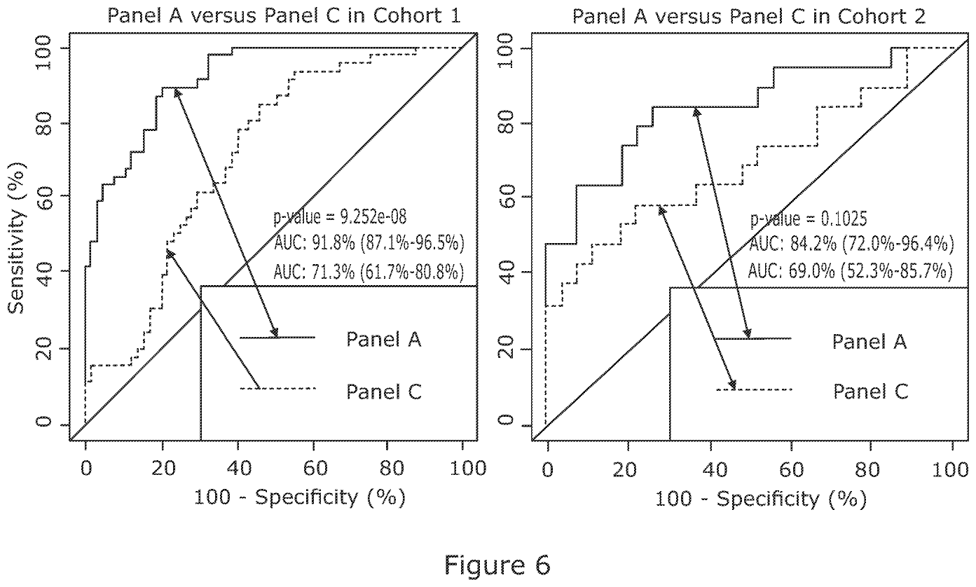

Panel A, comprises 13 biomarkers—SPATA19, TSPY3, GLS2, TCEA2, TSGA10, HMGN5, LUZP4, HDAC4, SPACA3, IMPDH1, TXN2, TFG and PPP2R1A (Table 2). This refined model was assessed in cohort 1 (AUC 0.918, Sensitivity 89.1%, Specificity 80.1%) and validated in the independent cohort 2 (AUC 0.842, Sensitivity 84.2%, Specificity 74.1%) (FIG. 4). There was no significant difference in the ROC metrics between the two cohorts, indicating good performance in the validation cohort. The strong CTAG presence in Panel A comprised six protein antigens (Panel C), SPATA19, SPACA3, TSPY3, TCEA2, TSGA10 and LUZP4, all with established pro-tumourigenic roles in cancer (Table 2). CTAGs trigger unprompted humoral immunity and immune responses in malignancies, altering tumour cell physiology and neoplastic behaviours. Their limited expression in normal somatic tissues coupled with recurrent up-regulation in epithelial carcinomas makes them highly attractive biomarker and vaccine targets. Performance of all three panels was explored in cohort 1 and 2 (FIGS. 5 and 6). However in cohort 2 (validation), the differences between panel A and the CTAG panels (B and C) was not significant. Panel B (16 CTAG panel) outperformed panel A in cohort 2 (AUC 0.875 versus 0.842, p=NS) but panel C underperformed compared to panel A in cohort 2 (AUC 0.69 versus 0.842, p=NS). The increased predictive performance of panel B (16 CTAG panel) reaffirms the importance of CTAGs in discriminating between survivorship in lung cancer. In spite of CTAG preponderance, this data shows that the non CTAG antigens in panel A, which are critical mediators of Wnt signalling and phosphatase activity are clearly biologically very important in their ability to prognosticate in lung cancer. Global score indexes were developed for each panel and the survival implications of each panel explored in multivariate models.

### Survival Analysis

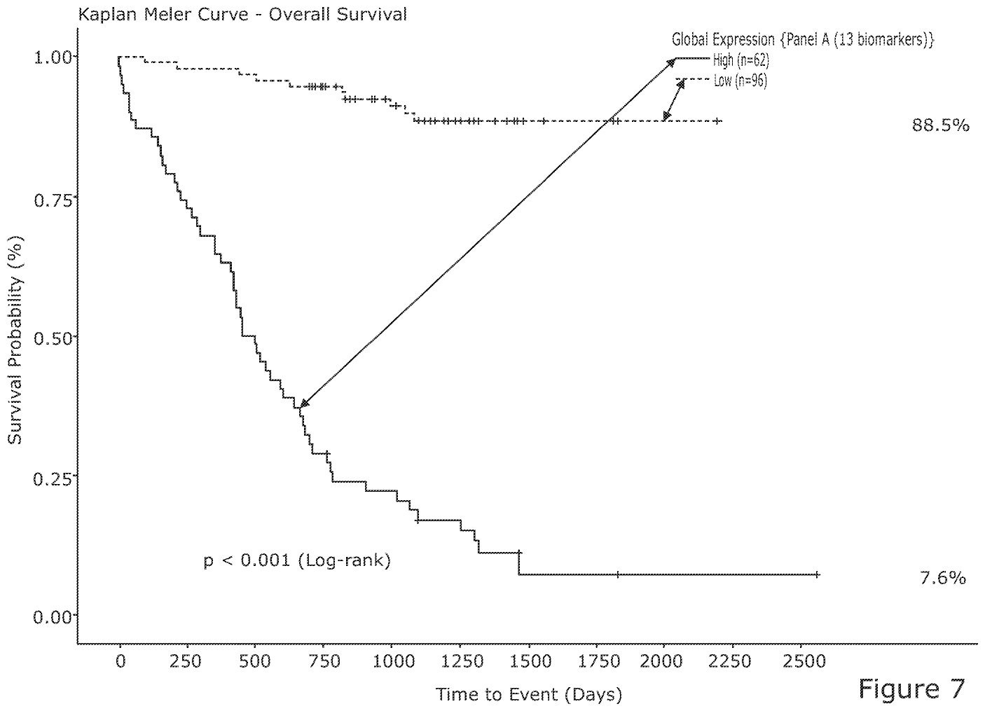

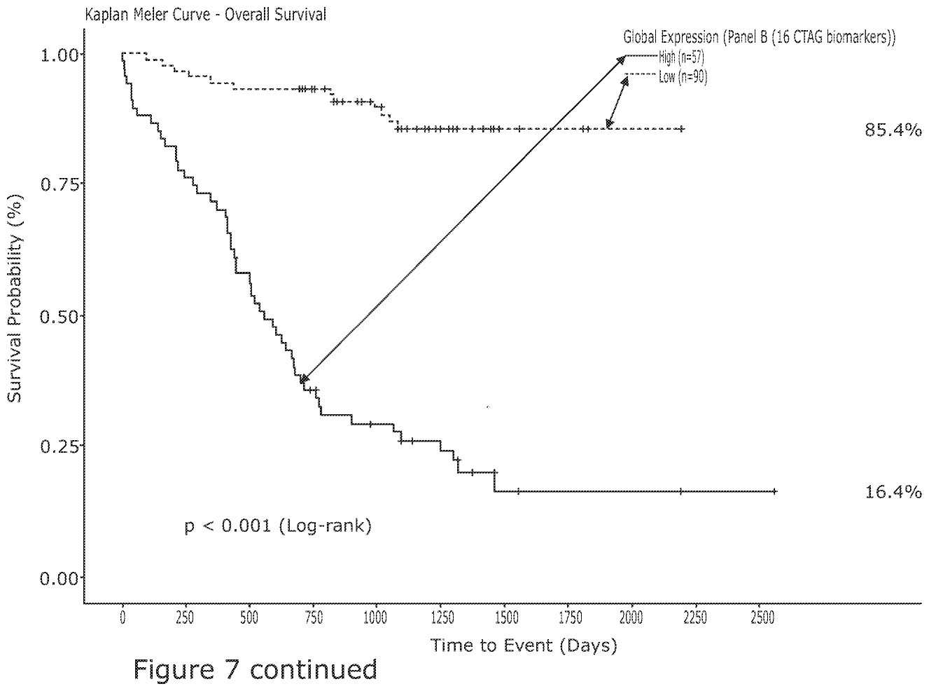

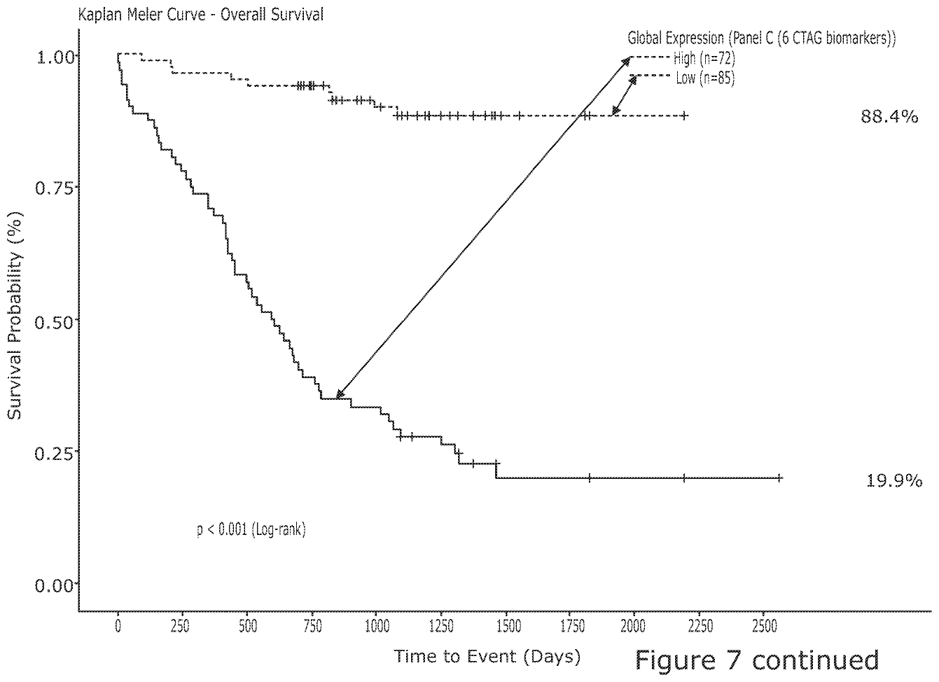

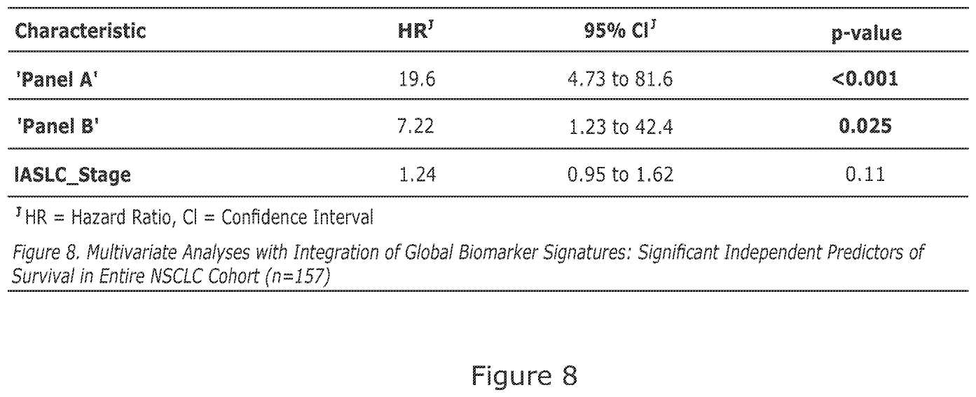

Further interrogation of these signatures was carried out by generating overall scores of the models for each patient. In order to further dichotomise between the samples (survivors and non-survivors), each panel was used to generate single “probability of outcome” scores for each patient. These scores were inferred directly from the biomarker signal intensities. Using an overall expression score for the panels, survival analyses were carried out (FIG. 7) and multivariate cox proportional hazards modelling (FIG. 8) in the entire NSCLC cohort. Patient age, gender, histology, nodal status, IASLC stage, lymphovascular invasion and whether patients underwent adjuvant chemotherapy were all predictors that were entered into the model alongside all the panel scores. All panels were able to effectively dichotomise between survivor statuses in the cohort, with high expression conferring a significantly worse outcome (p<0.001), reaffirming findings from the ROC analysis. Five year survival in high expressers of panels A, B and C was 7.6%, 16.4% and 19.9% respectively and high expressers of panel A had a median survival of just under 16 months which for early stage resected lung cancer is incredibly low. On multivariate testing, only panels A and B were deemed significant independent predictors of survival, HR 19.6 and 7.22 respectively (p<0.05). IASLC stage was still deemed an independent predictor of outcome albeit not significant (HR 1.24, p=0.11).

### Gene Ontology Analysis

Of the identified biomarkers in the RFE set (n=60), all are known for their role in biological processes heavily inter-twined with neoplasia and malignant transformation. Processes such as chromosomal organisation, cellular component homeostasis, ribosome function, transcription regulation, DNA repair and regulation of protein phosphotransferase activity, namely MAPK activation and the MAP/RAF kinase cascade (20) thus reaffirming their biological relevance. This is consistent with gene ontological analysis where the most significant pathways related to the RFE set, altered chromosome organisation [gene ontology (GO) term GO:0051276, false discovery rate (FDR)=5.24*10⁻³] and phosphotransferase activity (GO:0016776, FDR=3.89*10⁻²) in NSCLC.

Overall interaction enrichment for this selected number of biomarkers was significantly higher than would be expected for a random set of proteins of similar size, drawn from the genome (p=0.0022) suggesting biological interaction as a group and consistent with the preponderance of CTAGs. Further underscoring this; most of the seroreactive biomarkers are intracellular antigens (52/60) interacting with membrane and non-membrane bound organelles such as ribosomes (4/60) with the majority residing within the nucleus (37), a usually immuno-privileged site. This pattern has been observed in autoantibody studies in melanoma (14). Despite this, autoantibodies generated against autologous nuclear antigens are frequently found in cancer patient sera (13). Nuclear antigens however do not undergo antigen presentation during the negative selection of self-reactive lymphocytes largely because of their intrinsic proteolytic instability, which affects the binding kinetics with MHC class II receptors (13). Exposure of the nuclear antigens to one's immune system and the resultant generation of autoantibodies is therefore thought to occur following tumour cell death and release of the intracellular contents into the circulation (21).

### Discussion

Historically, the majority of autoantibody based biomarker research has concentrated on diagnosis of disease states or early detection of cancers as opposed to trying to map the course of disease post-treatment. This is true for NSCLC and melanoma (22). Overall, this balance is likely to shift in favour of the latter; results from the NLST and European NELSON trials (23,24), which favoured lung cancer screening, allied with an era of immunotherapy and checkpoint blockade is going to see more patients undergoing surgical resection for early stage disease and indeed more 10-based therapies for advanced disease.

In melanoma, autoantibodies have shown merit as prognostic biomarkers (25), however very few studies have detailed their efficacy. Rather than focus on a single uniquely predictive marker, antibody profiling offers high predictive power that is predicated on combining numerous tumour-associated antibodies. Given the complexity and multi-factorial nature of the anti-tumour immune response and tumour immune evasion mechanisms in cancers that are not solely reliant on single oncogenic drivers, combination biomarker signatures would prove more valuable. Meta-analytical data has further reinforced the need to devise multiple biomarker panels in order to deliver higher diagnostic potential in early lung cancer detection (26).

Using the approach adopted by Gnjatic and colleagues (12); identifying biomarkers based on RFU levels and positive seroreactivity in survivors versus non-survivors, 60 prognostic biomarkers were identified with individual ROC and survival data metrics. This collection of biomarkers demonstrates biological interaction as a group, partaking in key cellular processes that are often unregulated in tumours and are the inciting insult in tumorigenesis.

Most studies in the last decade that have explored serum or blood based antibodies targeting tumour-associated antigens have been for early lung cancer detection and have employed ELISA as the primary detection method, followed by Western blotting, protein chip and SDS-PAGE (26). Studies investigating single biomarkers have included Cyclin B1, p53, NY-ESO-1, MUC1, MDM2, p16, APE1, CD25, Cathepsin D, ABCC3, IGFBP-2, BARD1, BRAF, Dickkopf-1, c-Myc and a range of heat shock proteins (26). Sensitivities and specificities for lung cancer detection have ranged from 0-90.3% and 0 to 100% respectively, nicely demonstrated in a systematic review of proteomic signatures from 2019 (26). Studies investigating panels of biomarkers have commonly utilised p53, cyclin B1, MDM2, IMPDH, NY-ESO-1, CAGE, GAGE and MAGE family proteins, SOX2 and c-Myc. Sensitivities and specificities for lung cancer detection have ranged from 0-92.2% and 79.5 to 92.2% respectively. The models presented here predicted survivorship with an AUC of 0.875 and 0.842 in validation sets. This outperforms predictive capabilities of commonly used biomarkers in clinical practice such total PSA for prostate cancer (AUC 0.71) (27), pre-operative serum CEA for colorectal cancer (AUC 0.543) (28), NY-ESO-1 and Neuron-specific enolase (NSE) in small cell lung cancer (AUC 0.619 and 0.773 respectively) (29), and a panel of serum autoantibodies including NY-ESO-1, p53, MMP-7 and HSP70 in oesophageal adenocarcinoma (AUC 0.815) (30).

Much like the markers delineated in the dataset presented herein, these tumour-associated antigens are a combination of tumour suppressor genes and oncogenes, with roles in cell cycle regulation, DNA replication and apoptosis. These are processes that are commonly deregulated in various solid tumours such as breast, bladder, colon, oesophageal and prostate (26,31-33). Common biological themes from panel A include CTAG expression, Wnt signalling protein aberrancy and Serine/Threonine protein phosphatase deregulation.

CTAGs are united by their role in embryonic development and restriction of expression to male germ cells. Ectopic re-expression of these antigens has been seen in a variety of somatic solid tumours and in triple negative breast cancers, high expression correlated with worse survival in multivariate analysis (HR 2.02, 95% CI 1.27-3.20; p=0.003) (34). Ectopic gene signatures of normally silenced CTAG genes that are expressed in cancer too associated with a highly aggressive lung cancer phenotype and independently predicted poor outcome (35).

This has prompted their investigation as therapeutic targets and biomarkers of disease. Owing to their highly restricted expression patterns in normal tissues and ectopic expression in tumour types, their utility as individual diagnostic markers is limited but makes them highly sought after as targets for cancer vaccines (36).

The inventors identified 16 CTAGs (27%) in the RFE set as being highly discriminatory for survivorship in this distinct cohort of NSCLC patients (SPATA19, SPACA3, TSGA10, TSPY3, LUZP4, TCEA2, CTNNA2, MAGEB2, SPO11, MAGEB4, MAEL, CSAG1, MAGEB5, COX6B2, GAGE2, TSSK6). This CTAG-only model displayed high predictive power in the validation cohort (AUC 0.875, sensitivity 84.2%) and was a significant independent predictor of poor outcome.

A key element to the success of CTAG-dependent vaccine therapy is in appropriately identifying CTAG-expressing cancer cells that are abundant in tumours, rarely expressed in normal tissue, and have defined functional characteristics such that targeting results in the absolute attenuation of tumourigenic potential. Whilst peptide-based vaccine therapies alone have been met with challenges; MAGEA3 targeting although elicited CD8+ T cell clones, showed no measurable clinical benefit (40,44). Combining said therapies with immunogenic adjuvants, adoptive T cell transfer and even polyepitopic RNA based vaccines hold a lot of promise. The Lipo-MERIT trial demonstrated strong CD4+ and CD8+ T cell induction along with durable objective clinical benefit in unresectable melanoma patients treated with a poly-antigenic liposomal RNA vaccine with or without combination with anti-PD1 checkpoint blockade therapy (45). The RNA vaccine targeted four main CTAGs; NY-ESO-1, MAGEA3, TPTE and Tyrosinase (45). In the dataset presented herein, low CTAG expressers (FIG. 7) had good outcomes, with 85.4% 5-year overall survival (p<0.001), targeting this group is unlikely therefore to be of benefit, however high expressers who suffer poor outcomes post-resection may well be suitable for a CTAG-based polyepitopic RNA vaccine as an adjunct to standard adjuvant chemotherapy (Grabble et al., Nanomedicine, 2016, 11(20):2723-2734).

Cellular responses to DNA damage are integral to maintaining the genome and preventing cancer progression; Serine-Threonine phosphatases like Protein Phosphatase 2 play a key role in the DNA damage response through regulation of important cell cycle proteins and tumour suppressor genes such as ATM, Chk1, Chk2, p53 and BRCA1 (52). Cancer cells tend to evade the activation of DNA repair pathways through copy number alterations of Ser/Thr phosphatases, missense mutations and increased mutant gene expression. Identifying aberrancy of these important proteins and utilising early antigen expression is key to disease surveillance and therapeutics. Following the exploitation of BCR/ABL kinase inhibition in chronic myeloid leukaemia, efforts have been made to explore PP2A phosphatase reactivation/inhibition in anti-tumour therapy. PP2A complexes exert control over oncogenic signalling pathways (MEK/ERK, Srk-Jnk) and over collateral resistance phosphorylation pathways. Their inhibition in a KRAS-mutant human lung cancer cell line resulted in improved responses with MEK inhibitors (53). Current phase 2 trials in recurrent glioblastoma (Clinicaltrials.gov ID: NCT03027388) are investigating the role of PP2A inhibitor, LB100. TPTE, a CTAG also exerts PTEN-related tyrosine phosphatase activity and was one of the targets of the liposomal RNA vaccine used in the lipo-MERIT study (45) thus demonstrating the key role of protein phosphatases in tumorigenesis and how tumours exert oncogenic control through dysregulation of this proliferative brake.

The current era in lung cancer research utilises promising molecular biomarkers including auto-antibodies in the blood, complement fragments, circulating microRNAs, circulating tumour DNA, DNA methylation status of tumour tissue, direct profiling of tumour-associated antigens in serum and RNA airway and nasal signatures (3). Due to the sheer mass of biomarkers in need of clinical validation, standardised metrics of clinical utility are required as well as the use of newer AI-based and machine learning technologies to help select the most robust combinations.

Very few proteomic biomarker signatures in the current literature are powered to map disease post-treatment i.e. prognostic index, but instead they are used to diagnose disease by comparing with healthy patients. This unique dataset with its robust clinical stratification (death from recurrent disease in 12 months versus long term disease free survivorship at greater than 5 year follow-up) and long term follow-up is well placed to explore proteomic-based differences. This analysis utilised new machine learning approaches to derive unique biomarker signatures with the highest predictive capability in the patient cohorts. The biomarkers individually are highly relevant to cancer biology with important roles in key mechanisms that underlie tumorigenesis and are fueling current clinical trials in cancer medicine. These tightly regulated antigens with their almost uniform expression in epithelial carcinomas provide an excellent target for not only prognosticating disease but also as a therapeutic vaccine target, clearly exemplified by the data from the LipoMERIT study (45). The broad unsupervised interrogation of ≥1600 biomarkers in a robust clinical dataset only serves to reinforce this.

### REFERENCES

1. Ferlay J, Shin H-R, Bray F, Forman D, Mathers C, Parkin D M. Estimates of worldwide burden of cancer in 2008: GLOBOCAN 2008. Int J Cancer. 2010 Dec. 15; 127(12):2893-917.
2. International Early Lung Cancer Action Program Investigators, Henschke C I, Yankelevitz D F, Libby D M, Pasmantier M W, Smith J P, et al. Survival of patients with stage I lung cancer detected on C T screening. N Engl J Med. 2006 Oct. 26; 355(17):1763-71.
3. Seijo L M, Peled N, Ajona D, Boeri M, Field J K, Sozzi G, et al. Biomarkers in Lung Cancer Screening: Achievements, Promises, and Challenges. J Thorac Oncol Off Publ Int Assoc Study Lung Cancer. 2019 Mar. 14(3):343-57.
4. Burotto M, Thomas A, Subramaniam D, Giaccone G, Rajan A. Biomarkers in Early-Stage Non-Small-Cell Lung Cancer: Current Concepts and Future Directions. J Thorac Oncol. 2014 Nov. 9(11):1609-17.
5. Vargas A J, Harris C C. Biomarker development in the precision medicine era: lung cancer as a case study. Nat Rev Cancer. 2016 Aug. 16(8):525-37.
6. Jamal-Hanjani M, Wilson G A, McGranahan N, Birkbak N J, Watkins TBK, Veeriah S, et al. Tracking the Evolution of Non-Small-Cell Lung Cancer. N Engl J Med. 2017 Jun. 1; 376(22):2109-21.
7. Xin L, Liu Y-H, Martin T A, Jiang W G. The Era of Multigene Panels Comes? The Clinical Utility of Oncotype D X and MammaPrint. World J Oncol. 2017 Apr. 8(2):34-40.
8. Roberts M C, Weinberger M, Dusetzina S B, Dinan M A, Reeder-Hayes K E, Carey L A, et al. Racial Variation in the Uptake of Oncotype D X Testing for Early-Stage Breast Cancer. J Clin Oncol. 2016 Jan. 10; 34(2):130-8.
9. Shinjo K, Okamoto Y, An B, Yokoyama T, Takeuchi I, Fujii M, et al. Integrated analysis of genetic and epigenetic alterations reveals CpG island methylator phenotype associated with distinct clinical characters of lung adenocarcinoma. Carcinogenesis. 2012 July; 33(7):1277-85.
10. Govindan R, Ding L, Griffith M, Subramanian J, Dees N D, Kanchi K L, et al. Genomic landscape of non-small cell lung cancer in smokers and never-smokers. Cell. 2012 Sep. 14; 150(6):1121-34.
11. Yanagisawa K, Tomida S, Shimada Y, Yatabe Y, Mitsudomi T, Takahashi T. A 25-signal proteomic signature and outcome for patients with resected non-small-cell lung cancer. J Natl Cancer Inst. 2007 Jun. 6; 99(11):858-67.
12. Gnjatic S, Wheeler C, Ebner M, Ritter E, Murray A, Altorki N K, et al. Seromic analysis of antibody responses in non-small cell lung cancer patients and healthy donors using conformational protein arrays. J Immunol Methods. 2009 Feb. 28; 341(1-2):50-8.
13. Zaenker P, Gray E S, Ziman M R. Autoantibody Production in Cancer—The Humoral Immune Response toward Autologous Antigens in Cancer Patients. Autoimmun Rev. 2016 May; 15(5):477-83.
14. Zaenker P, Lo J, Pearce R, Cantwell P, Cowell L, Lee M, et al. A diagnostic autoantibody signature for primary cutaneous melanoma. Oncotarget. 2018 Jul. 17; 9(55):30539-51.
15. Ramachandran N, Raphael J V, Hainsworth E, Demirkan G, Fuentes M G, Rolfs A, et al. Next-generation high-density self-assembling functional protein arrays. Nat Methods. 2008 Jun. 5(6):535-8.
16. Kuhn M, Johnson K. Over-Fitting and Model Tuning. In: Kuhn M, Johnson K, editors. Applied Predictive Modeling [Internet]. New York, NY: Springer; 2013 [cited 2021 Mar. 6]. p. 61-92. Available from: https://doi.org/10.1007/978-1-4614-6849-3_4
17. Dobbin K K, Simon R M. Optimally splitting cases for training and testing high dimensional classifiers. BMC Med Genomics. 201j1 April; 8; 4(1):31.
18. Kuhn M. Building predictive models in R using the caret package. J Stat Softw. 2008; 28(5):1-26.
19. Kai-Bo Duan, Rajapakse J C, Haiying Wang, Azuaje F. Multiple SVM-RFE for gene selection in cancer classification with expression data. IEEE Trans NanoBioscience. 2005 Sep. 4(3):228-34.
20. Jeggo P A, Pearl L H, Carr A M. DNA repair, genome stability and cancer: a historical perspective. Nat Rev Cancer. 2016 Jan. 16(1):35-42.
21. Carl P L, Temple BRS, Cohen P L. Most nuclear systemic autoantigens are extremely disordered proteins: implications for the etiology of systemic autoimmunity. Arthritis Res Ther. 2005; 7(6):R1360-1374.
22. Gray E S, Rizos H, Reid A L, Boyd S C, Pereira M R, Lo J, et al. Circulating tumor DNA to monitor treatment response and detect acquired resistance in patients with metastatic melanoma. Oncotarget. 2015 Dec. 8; 6(39):42008-18.
23. Yousaf-Khan U, van der Aalst C, de Jong P A, Heuvelmans M, Scholten E, Lammers J-W, et al. Final screening round of the NELSON lung cancer screening trial: the effect of a 2.5-year screening interval. Thorax. 2017 January; 72(1):48-56.
24. Horeweg N, Scholten E T, de Jong P A, van der Aalst C M, Weenink C, Lammers J-W J, et al. Detection of lung cancer through low-dose C T screening (NELSON): a prespecified analysis of screening test performance and interval cancers. Lancet Oncol. 2014 Nov. 15(12):1342-50.
25. Zornig I, Halama N, Lorenzo Bermejo J, Ziegelmeier C, Dickes E, Migdoll A, et al. Prognostic significance of spontaneous antibody responses against tumor-associated antigens in malignant melanoma patients. Int J Cancer. 2015 Jan. 1; 136(1):138-51.
26. Yang B, Li X, Ren T, Yin Y. Autoantibodies as diagnostic biomarkers for lung cancer: A systematic review. Cell Death Discov. 2019 Dec. 5(1):126.
27. Oto J, Fernández-Pardo Á, Royo M, Hervás D, Martos L, Vera-Donoso C D, et al. A predictive model for prostate cancer incorporating PSA molecular forms and age. Sci Rep. 2020 Feb. 12; 10(1):2463.
28. Huang C-S, Chen C-Y, Huang L-K, Wang W-S, Yang S-H. Prognostic value of postoperative serum carcinoembryonic antigen levels in colorectal cancer patients who smoke. PLOS ONE. 2020 Jun. 5; 15(6):e0233687.
29. Yang J, Jiao S, Kang J, Li R, Zhang G. Application of serum NY-ESO-1 antibody assay for early SCLC diagnosis. Int J Clin Exp Pathol. 2015; 8(11):14959-64.
30. Xu Y-W, Chen H, Guo H-P, Yang S-H, Luo Y-H, Liu C-T, et al. Combined detection of serum autoantibodies as diagnostic biomarkers in esophagogastric junction adenocarcinoma. Gastric Cancer. 2019 May 1; 22(3):546-57.
31. Zhao J, Wang Y, Wu X. HMGN5 promotes proliferation and invasion via the activation of Wnt/β-catenin signaling pathway in pancreatic ductal adenocarcinoma. Oncol Lett. 2018 Sep. 16(3):4013-9.
32. Wu J, Wang J. HMGN5 expression in bladder cancer tissue and its role on prognosis. Eur Rev Med Pharmacol Sci. 2018 Feb. 22(4):970-5.
33. Li Q, Wei P, Huang B, Xu Y, Li X, Li Y, et al. MAEL expression links epithelial-mesenchymal transition and stem cell properties in colorectal cancer. Int J Cancer. 2016 Dec. 1; 139(11):2502-11.
34. Karn T, Pusztai L, Ruckhaberle E, Liedtke C, Muller V, Schmidt M, et al. Melanoma antigen family A identified by the bimodality index defines a subset of triple negative breast cancers as candidates for immune response augmentation. Eur J Cancer Oxf Engl 1990. 2012 January; 48(1):12-23.
35. Rousseaux S, Debernardi A, Jacquiau B, Vitte A-L, Vesin A, Nagy-Mignotte H, et al. Ectopic activation of germline and placental genes identifies aggressive metastasis-prone lung cancers. Sci Transl Med. 2013 May 22; 5(186):186ra66.
36. Simpson A J G, Caballero O L, Jungbluth A, Chen Y-T, Old L J. Cancer/testis antigens, gametogenesis and cancer. Nat Rev Cancer. 2005 Aug. 5(8):615-25.
37. Jakobsen M K, Gjerstorff M F. CAR T-Cell Cancer Therapy Targeting Surface Cancer/Testis Antigens. Front Immunol [Internet]. 2020 [cited 2021 Feb. 17]; 11. Available from: https://www.frontiersin.org/articles/10.3389/fimmu.2020.01568/full
38. Raza A, Merhi M, Inchakalody V P, Krishnankutty R, Relecom A, Uddin S, et al. Unleashing the immune response to NY-ESO-1 cancer testis antigen as a potential target for cancer immunotherapy. J Transl Med. 2020 Mar. 27; 18(1):140.
39. Qiu C-X, Bai X-F, Shen Y, Zhou Z, Pan L-Q, Xu Y-C, et al. Specific Inhibition of Tumor Growth by T Cell Receptor-Drug Conjugates Targeting Intracellular Cancer-Testis Antigen NY-ESO-1/LAGE-1. Bioconjug Chem. 2020 Dec. 16; 31(12):2767-78.
40. Li X-F, Ren P, Shen W-Z, Jin X, Zhang J. The expression, modulation and use of cancer-testis antigens as potential biomarkers for cancer immunotherapy. Am J Transl Res. 2020 Nov. 15; 12(11):7002-19.
41. Fanjul-Fernández M, Quesada V, Cabanillas R, Cadiñanos J, Fontanil T, Obaya A, et al. Cell-cell adhesion genes CTNNA2 and CTNNA3 are tumour suppressors frequently mutated in laryngeal carcinomas. Nat Commun. 2013; 4:2531.
42. McGranahan N, Favero F, de Bruin E C, Birkbak N J, Szallasi Z, Swanton C. Clonal status of actionable driver events and the timing of mutational processes in cancer evolution. Sci Transl Med. 2015 Apr. 15; 7(283):283ra54.
43. Fratta E, Coral S, Covre A, Parisi G, Colizzi F, Danielli R, et al. The biology of cancer testis antigens: putative function, regulation and therapeutic potential. Mol Oncol. 2011 Apr. 5(2):164-82.
44. Vansteenkiste J F, Cho B C, Vanakesa T, De Pas T, Zielinski M, Kim M S, et al. Efficacy of the MAGE-A3 cancer immunotherapeutic as adjuvant therapy in patients with resected MAGE-A3-positive non-small-cell lung cancer (MAGRIT): a randomised, double-blind, placebo-controlled, phase 3 trial. Lancet Oncol. 2016 Jun. 17(6):822-35.
45. Sahin U, Oehm P, Derhovanessian E, Jabulowsky R A, Vormehr M, Gold M, et al. An RNA vaccine drives immunity in checkpoint-inhibitor-treated melanoma. Nature. 2020 September; 585(7823):107-12.
46. Wang Z, Li Z, Ji H. Direct targeting of 0-catenin in the Wnt signaling pathway: Current progress and perspectives. Med Res Rev [Internet]. [cited 2021 Feb. 1];n/a(n/a). Available from: https://onlinelibrary.wiley.com/doi/abs/10.1002/med.21787
47. Kim J-H, Kwon J, Lee H W, Kang M C, Yoon H-J, Lee S-T, et al. Protein tyrosine kinase 7 plays a tumor suppressor role by inhibiting ERK and AKT phosphorylation in lung cancer. Oncol Rep. 2014 Jun. 1; 31(6):2708-12.
48. Semenov M V, Tamai K, Brott B K, Kühl M, Sokol S, He X. Head inducer Dickkopf-1 is a ligand for Wnt coreceptor LRP6. Curr Biol C B. 2001 Jun. 26; 11(12):951-61.
49. Zhang X, Ning Y, Xiao Y, Duan H, Qu G, Liu X, et al. MAEL contributes to gastric cancer progression by promoting ILKAP degradation. Oncotarget. 2017 Dec. 26; 8(69):113331-44.
50. Damelin M, Bankovich A, Bernstein J, Lucas J, Chen L, Williams S, et al. A PTK7-targeted antibody-drug conjugate reduces tumor-initiating cells and induces sustained tumor regressions. Sci Transl Med. 2017 Jan. 11; 9(372):eaag2611.
51. Gurney A, Axelrod F, Bond C J, Cain J, Chartier C, Donigan L, et al. Wnt pathway inhibition via the targeting of Frizzled receptors results in decreased growth and tumorigenicity of human tumors. Proc Natl Acad Sci USA. 2012 Jul. 17; 109(29):11717-22.
52. Peng A, Maller J L. Serine/threonine phosphatases in the DNA damage response and cancer. Oncogene. 2010 Nov. 29(45):5977-88.
53. Kauko 0, O'Connor C M, Kulesskiy E, Sangodkar J, Aakula A, Izadmehr S, et al. PP2A inhibition is a druggable MEK inhibitor resistance mechanism in KRAS-mutant lung cancer cells. Sci Transl Med. 2018 Jul. 18; 10(450).
54. Detterbeck F C, Boffa D J, Kim A W, Tanoue L T. The Eighth Edition Lung Cancer Stage Classification. Chest. 2017 January; 151(1):193-203.
55. Rathinam S, Ward D G, James N D, Rajesh P B. Proteomic analysis of resectable non-small cell lung cancer: post-resection serum samples may be useful in identifying potential markers. Interact Cardiovasc Thorac Surg. 2011 Jul. 13(1):3-6.
56. Sumera A, Anuar N D, Radhakrishnan A K, Ibrahim H, Rutt N H, Ismail N H, et al. A Novel Method to Identify Autoantibodies against Putative Target Proteins in Serum from beta-Thalassemia Major: A Pilot Study. Biomedicines. 2020 Apr. 26; 8(5).
57. Duarte J, Serufuri J-M, Mulder N, Blackburn J. Protein Function Microarrays: Design, Use and Bioinformatic Analysis in Cancer Biomarker Discovery and Quantitation. In: Wang X, editor. Bioinformatics of Human Proteomics [Internet]. Dordrecht: Springer Netherlands; 2013 [cited 2021 Mar. 6]. p. 39-74. (Translational Bioinformatics). Available from: https://doi.org/10.1007/978-94-007-5811-7_3
58. Bommert A, Sun X, Bischl B, Rahnenführer J, Lang M. Benchmark for filter methods for feature selection in high-dimensional classification data. Comput Stat Data Anal. 2020 March; 143.
59. Wright M N, Ziegler A. Ranger: A fast implementation of random forests for high dimensional data in C++ and R. J Stat Softw. 2017; 77(1).
60. Tibshirani R. Regression Shrinkage and Selection via the Lasso. J R Stat Soc Ser B Methodol. 1996; 58(1):267-88.
61. Multimodel Inference: Understanding AIC and BIC in Model Selection—Kenneth P. Burnham, David R. Anderson, 2004 [Internet]. [cited 2021 Mar. 6]. Available from: https://journals.sagepub.com/doi/10.1177/0049124104268644
62. Akaike H. Information Theory and an Extension of the Maximum Likelihood Principle. In: Parzen E, Tanabe K, Kitagawa G, editors. Selected Papers of Hirotugu Akaike [Internet]. New York, NY: Springer; 1998 [cited 2021 Mar. 6]. p. 199-213. (Springer Series in Statistics). Available from: https://doi.org/10.1007/978-1-4612-1694-0_15
63. Cooper J D, Han S Y S, Tomasik J, Ozcan S, Rustogi N, van Beveren N J M, et al. Multimodel inference for biomarker development: an application to schizophrenia. Transl Psychiatry. 2019 Feb. 11; 9(1):1-10.
64. López-Ratón M, Rodriguez-Alvarez M X, Suárez C C, Sampedro F G. OptimalCutpoints: An R Package for Selecting Optimal Cutpoints in Diagnostic Tests. J Stat Softw. 2014; 61(8).
65. Wickham H. ggplot2. Chain: Springer International Publishing; 2016.
66. Kassambara A, Kosinski M, Biecek P, others. survminer: Drawing Survival Curves using′ggplot2′. R Package Version 03. 2017; 1.
67. Bischl B, Lang M, Kotthoff L, Schiffner J, Richter J, Studerus E, et al. Mlr: Machine Learning in R. J Mach Learn Res. 2016 Jan. 17(1):5938-5942.
68. Strobl C, Boulesteix A L, Kneib T, Augustin T, Zeileis A. Conditional variable importance for random forests. BMC Bioinformatics. 2008 Dec. 11; 9(1).
69. Breiman L. Random forests. Mach Learn. 2001; 45(1):5-32.
70. Kursa M B. praznik: Tools for Information-Based Feature Selection. 2020.
71. Qiu W. Sample Size and Power Calculation in Microarray Studies Using the sizepower package:8.
72. Scherer R. shearer/samplesize [Internet]. 2019 [cited 2021 Mar. 6]. Available from: https://github.com/shearer/samplesize

1. A method of prognosing a subject, the method comprising:
i. providing a biological sample obtained from the subject;
ii. determining the level of a panel of biomarkers comprising or consisting of SPATA19, SPACA3, TSPY3, TCEA2, TSGA10, and LUZP4 in the sample;
iii. comparing the level of the panel of biomarkers in the sample with a reference level of the same panel of biomarkers;
iv. using the results from (iii) to prognose the subject.

2. The method of claim 1, wherein the subject has been diagnosed with cancer.

3. The method of claim 1 or claim 2, wherein the reference level of the same panel of biomarkers is determined in a biological sample obtained from a patient who at 5 years post-surgical resection is alive and determined to be cancer-free.

4. The method of any of claims 1-3, wherein the prognosis is the likelihood of survival.

5. The method of any of claims 1-4, wherein the likelihood of survival is given as the likelihood of surviving 5 years or more after the prognosis.

6. The method of any of claims 1-5, wherein the subject has a low likelihood of survival when the level of the panel of biomarkers is high.

7. The method of any of claims 1-6, wherein when the biomarker panel consists of SPATA19, SPACA3, TSPY3, TCEA2, TSGA10, and LUZP4, the level of the biomarkers is determined to be low when a ROC value of below around 40 to 45 is calculated and;
the level of the level of the panel is determined to be high when a ROC value of over around 40 to 45 is calculated.

8. The method of any of claims 1-6, wherein the panel further comprises or consist of one or more of, such as one of, two of, three of, four of, five of, six of, or all of GLS2, HMGN5, HDAC4, IMPDH1, TXN2, TFG, and PPP2R1A.

9. The method of claim 8, wherein when the biomarker panel consists of SPATA19, SPACA3, TSPY3, TCEA2, TSGA10, LUZP4, GLS2, HMGN5, HDAC4, IMPDH1, TXN2, TFG, and PPP2R1A, the level of the biomarker panel is determined to be low when a ROC value of below around 60 to 70 is calculated, and the level of the level of the biomarker panel is determined to be high when a ROC value of over around 60 to 70 is calculated.

10. The method of any of claims 1-6, wherein the panel further comprises or consist of one or more of, such as one of, two of, three of, four of, five of, six of, seven of, eight of, nine of, or all of CTNNA2, MAGEB2, SPO11, MAGEB4, MAEL, CSAG1, MAGEB5, COX6B2, GAGE2, or TSSK6.

11. The method of claim 10, wherein when the biomarker panel consists of SPATA19, SPACA3, TSPY3, TCEA2, TSGA10 LUZP4, CTNNA2, MAGEB2, SPO11, MAGEB4, MAEL, CSAG1, MAGEB5, COX6B2, GAGE2, and TSSK6, the level of the panel is determined to be low when a ROC value of below around 40 to 50 is calculated, and the level of the panel is determined to be high when a ROC value of above around 40 to 50 is calculated.

12. The method of any of claims 1-6, wherein the panel further comprises or consists of one or more of, such as one of, two of, three of, four of, five of, six of, seven of, eight of, nine of, ten of, 11 of, 12 of, 13 of, 14 of, 15 of, 16 of, 17 of, 18 of, 19 of, 20 of, 21 of, 22 of, 23 of, 24 of, 25 of, 26 of, 27 of, 28 of, 29 of, 30 of, 31 of, 32 of, 33 of, 34 of, 35 of, 36 of, 37 of, 38 of, 39 of, 40 of, 41 of, 42 of, 43 of, 44 of, 45 of, 46 of, 47 of, 48 of, 49 of, 50 of, 51 of, 52 of, 53 of, or all of CASP7, GLS2, CTNNA2, ITPKB, AFF4, MAGEB2, C1orf174, TYRO3_int, DCBLD2, PCLAF, SPO11, BPIFA1, MAGEB4, HMGN5, MAEL, HDAC4, SOX15, HOOK1, CDK16, CSAG1, IMPDH1, MAGEB5, TXN2, NFYA, PHF7, HIST1H1C, IP6K1, TFG, AIM2, SGO1, PYCR1, FAM50B, HK2, ERBB3_int, TBL1X, ZNF207, EEF1D, PPP2R1A, MAP2K7, RPL7A, CBLC, COX6B2, ACTB, CA9, FLCN, GAGE2, ARAF, AK3, HMG20B, CNN1, EPAS1, EAPP, TSSK6, and GRK6.

13. A method of identifying a subject likely to benefit from treatment, the method comprising:
i. performing the method of any of claims 1-12; and
ii. determining that a subject with a poor prognosis is likely to benefit from treatment.

14. The method of any of claims 1-13, wherein the sample is a blood sample.

15. The method of any of claims 1-14, wherein the step of determining the level of a panel of biomarkers comprises determining the level of autoantibodies in the sample which specifically recognise each biomarker in the panel.

16. The method of any of claims 1-15, further comprising administering a therapeutic medication to the subject if the individual is given a poor prognosis or is identified as likely to benefit from treatment.

17. The method of claim 16, wherein the therapeutic medication comprises of consists of one or more of the panel, one or more nucleic acid such as DNA or mRNA which encodes one or more of the panel, or a nucleic acid such as DNA or mRNA which encodes an antibody which recognises one or more of the panel.

18. A pharmaceutical composition comprising:
(i) one or more of, such as one, two, three, four, five, or six of SPATA19, SPACA3, TSPY3, TCEA2, TSGA10, and LUZP4; or
(ii) one or more nucleic acid such as DNA or mRNA which encodes one or more of, such as one, two, three, four, five, or six of SPATA19, SPACA3, TSPY3, TCEA2, TSGA10, and LUZP4; or
(iii) one or more nucleic acid such as DNA or mRNA which encodes an antibody which recognises one or more of, such as one, two, three, four, five, or six of SPATA19, SPACA3, TSPY3, TCEA2, TSGA10, and LUZP4.

19. A method of treating and/or preventing lung cancer, such as NSCLC, in a subject, the method comprising:
administering a therapeutically effective amount of the pharmaceutical composition of claim 18 to the subject.

20. A kit comprising probes capable of binding to each of a panel of biomarkers comprising or consisting of SPATA19, SPACA3, TSPY3, TCEA2, TSGA10, and LUZP4, optionally wherein the kit further comprises a set of instructions.

21. A kit comprising probes capable of binding to autoantibodies which specifically recognise each of a panel of biomarkers comprising or consisting of SPATA19, SPACA3, TSPY3, TCEA2, TSGA10, and LUZP4, optionally wherein the kit further comprises a set of instructions.
